(19) United States
(12) Patent Application Publication
(10) Pub. No.: US 2009/0142301 A1
(43) Pub. Date: Jun. 4, 2009

(54) NOVEL PHARMACEUTICAL COMPOSITION OF INTERFERON GAMMA OR PIRFENIDONE WITH MOLECULAR DIAGNOSTICS FOR THE IMPROVED TREATMENT OF INTERSTITIAL LUNG DISEASES

(76) Inventors: Dorian Bevec, Gentilino (CH); Rolf Ziesche, Wein (AT)

Correspondence Address: WINSTEAD PC, P.O. BOX 50784, DALLAS, TX, 75201, US

(21) Appl. No.: 12/110,151

(22) Filed: Apr. 25, 2008

Related U.S. Application Data
(63) Continuation of application No. 10/498,079, filed on May 5, 2006, now abandoned.

(30) Foreign Application Priority Data
Dec. 18, 2001 (EP) 01130011.8

(51) Int. Cl. A61K 38/21; C12Q 1/68; C12Q 1/48; C12Q 1/40; A61K 31/445; C12Q 1/26; C12Q 1/42; C40B 30/04

(52) U.S. Cl. 424/85.5; 435/6; 435/15; 435/22; 435/25; 435/21; 506/9; 514/317

(57) ABSTRACT

The present invention relates to a novel pharmaceutical composition of compounds having the biological activity of interferon gamma (IFN-γ) or pirfenidone in combination with a diagnostic array of candidate polynucleotides for the improved treatment of all forms of interstitial lung disease, in particular of idiopathic pulmonary fibrosis (IPF).

### CROSS-REFERENCE TO RELATED APPLICATIONS

This application is a continuation of U.S. patent application Ser. No. 10/498,079 filed May, 05, 2006, which is a 35 U.S.C. 371 national application of International Application Number PCT/CH02/00691 filed on Dec. 12, 2002, which designated the United States, and further, such applications are hereby incorporated by reference.

### BACKGROUND OF THE INVENTION

The present invention relates to a novel pharmaceutical composition of compounds having the biological activity of interferon gamma (IFN-γ) or pirfenidone in combination with a diagnostic array of candidate polynucleotides for the improved treatment of all forms of interstitial lung diseases, in particular of idiopathic pulmonary fibrosis (IPF).

### Interstitial Lung Diseases

Reliable diagnosis of is of critical importance for disease management, research, epidemiology and development of specific therapies.

Interstitial lung diseases (ILD) are a heterogeneic group of chronic inflammatory reactions of the lung. Different forms of ILD are known which comprise, for example, of idiopathic pulmonary fibrosis (IPF), hypersensivity pneumonitis, scleroderma, Systemic Lupus Erythematosus, Rheumatoid Arthritis, Churg-Strauss syndrome, Wegener's granulomatosis, and Goodpasture Syndrome. This process is characterized by a combination of injury and exaggerated but fuitile attempts of tissue repair that transforms the regular maintenance of cellular growth into progressive development of scars. Characteristic features of this reaction are: repeated damage; intensified proteolytic activity and change in the composition of extra-cellular matrix (ECM) components. This combination leads to a shifted cellular immune response and a relentless induction of mesenchymal growth.

Cytokines, such as tumor necrosis factor-α, and mediators of growth, such as transforming growth factor β₁ (TGFβ₁), have long been implicated in this process. Of these mediators, TGFβ₁, has probably become the most important one, due to its strong activity to stimulate mesenchymal growth and its ability to modulate cellular immune functions. TGF-β₁ is known to cause severe pulmonary fibrosis when overexpressed in animal models (Sime P J, Xing Z. Graham F L, Csaky K G, Gauldie J. Adenovector-mediated gene transfer of active transforming growth factor β₁ induces prolonged severe fibrosis in rat lung. J Clin Invest 1997; 100:768-76.), and a significant overexpression of this mediator is found in human pulmonary fibrosis (Broekelmann T J, Limper A H, Colby T V, McDonald J A. Transforming growth factor beta 1 is present at sites of extracellular matrix gene expression in human pulmonary fibrosis. Proc Natl Acad Sci USA 1991; 88:6642-6.). The immunomodulatory action of TGF-β₁ is well-known (Qin L, Ding Y, Bromberg J S. Gene transfer of transforming growth factor-beta 1 prolongs murine cardiac allograft survival by inhibiting cell-mediated immunity. Hum Gene Ther 1996; 7:1981-8; Gilbert K M, Thoman M, Bauche K, Pham T. Weigle W O. Transforming growth factor-beta 1 induces antigen-specific unresponsiveness in naive T cells. Immunol Invest 1997; 26:459-72; Lawrence D A. Transforming growth factor-beta: a general review. Eur Cytokine Netw 1996; 7:363-74.). It includes the inhibition of interferon gamma release (Naganuma H, Sasaki A, Satoh E, Nagasaka M, Nakano S, Isoe S, et al. Transforming growth factor-beta inhibits interferon gamma secretion by lymphokine-activated killer cells stimulated with tumor cells. Neurol Med Chir Tokyo 1996; 36: 789-95.), the suppression of interferon gamma-dependent immune reactions (Lee Y J, Han Y, Lu H T, Nguyen V, Qin H, Howe P H, et al. TGF-beta suppresses interferon gamma induction of class II MHC gene expression by inhibiting class II transactivator messenger RNA expression. J Immunol 1997; 158:2065-75.), and the induction of immunosuppressive CD8+ lymphocytes (Gray J D, Hirokawa M, Horwitz D A, J Exp Med 180, 1937-1942, 1994.). Indeed, modulation of cellular immunity in patients with progressive fibrosis has been observed for years, even in forms, which clearly represent different mechanisms of initial damage. Recent investigations have demonstrated that progressive scarring in idiopathic pulmonary fibrosis is accompanied by a shift of the cytokine balance in T lymphocytes that favors the formation of the so-called T helper type 2 (Th2) reaction (Prior C, Haslam P L. In vivo levels and in vitro production of interferon gamma in fibrosing interstitial lung diseases. Clin Exp Immunol 1992; 88:280-7.; Ziesche R, Kink E, Herold C, Podolsky A, Block L H. Therapy of chronic interstitial lung disease with a combination of Interferon gamma and low-dose prednisolone Chest 1996; 110:255.). This reaction is characterized by an increase of ‘Th2’ cytokines, such as interleukin (IL)-4, IL-10 and IL-13, and a reduction or even a complete loss of interferon gamma, the main mediator of the T helper type 1 reaction (Majumdar S, Li D, Ansari T. et al. Tissue cytokine profiles of cryptogenic fibrosing alveolitis (CFA) and fibrosing alveolitis associated with systemic sclerosis (FASSc) are distinct: a quantitative in situ study of open lung biopsies. Eur Respir J 1999; 14: 251-7.). In bleomycin-induced lung fibrosis, transcription of the interferon gamma gene decreases from day 7 onwards and is no longer detectable on day 28. Reciprocally, transcription of IL-13, which also has fibrogenic activity, (Romagnani S. Th1/Th2 cells. Inflamm Bowel Dis. 1999; 5: 285-94; Fallon P G, Richardson E J, McKenzie G J, McKenzie A N. Schistosome infection of transgenic mice defines distinct and contrasting pathogenic roles for IL-4 and IL-13: IL-13 is a profibrotic agent. J Immunol. 2000; 164:2585-91.), begins to increase on day 7. In contrast to the chronic inflammatory reaction in fibrosis, acute inflammation of the pulmonary interstitium as a result of infection with M. pneumoniae is characterized by a simultaneous transcription of both Th1 and Th2 cytokine genes, i.e. IL-12 and interferon gamma, and IL-4. In addition, this reaction includes an increased transcription of TGF-β₁, probably as a sign of activated tissue repair.

Unfortunately, as a result of the usually late recognition of IPF, the early cellular events in this disease are virtually impossible to assess. However, analysis of mRNA accumulation for IL-4, interferon gamma, and TGF-β₁ in an individual with familial idiopathic pulmonary fibrosis, with symptoms of breathlessness on maximum exertion of less than a year, already demonstrates the shift of the immune balance and the intense activation of transcription of TGF-β₁. In the group of individuals with idiopathic pulmonary fibrosis, we found that the amount of mRNA accumulation of the TGF-β₁ gene was seven fold higher than that in normal lungs. In conclusion, our observations clearly demonstrate features of pathologically intensified repair mechanisms.

The intensity of repair is directly influenced by mechanisms of inflammation causing intensified turnover of both ECM and cellular components. As a result, chronic inflammation, usually induced by various infective agents, aggravates pathologic tissue repair.

Even if the causative agents were known, at present organ fibrosis cannot be sufficiently treated with any medication. Millions of people are dying from slow destruction of vital organ systems owing to pathological restructuring of functional organ tissue. This relentless process is known as fibrosis or tissue remodeling that is primarily due to fibroproliferative mechanisms. The only remedy is organ transplantation, which is associated with numerous costly complications.

The fibroproliferative reaction concerns all organs of the body. In the gas-exchanging lung tissue it is known as lung fibrosis, in the bronchi it is known as bronchial asthma, in the liver as cirrhosis, in the kidney as glomerulosclerosis, in the general circulation as arteriosclerosis and coronary artery sclerosis, and in the pulmonary circulation as pulmonary hypertension.

These conditions are collectively known as fibroproliferative diseases. In fibrosis, healthy tissue is progressively replaced by components of the connective and supporting tissue of the human body. This process is based on the pathologically accelerated growth rate of tissue cells, which would normally accomplish regular wound healing. Thus, fibroproliferative diseases may be defined as uncontrollably accelerated wound healing. In fibrosis, the replacement of functional organ tissue continues until complete loss of organ function occurs. The currently available modes of diagnosis and treatment for all fibroproliferative diseases are inadequate.

### Pirfenidone

Pirfenidone is an orally active small molecule drug that appears to inhibit collagen synthesis, down regulates production of multiple cytokines and blocks fibroblast proliferation and stimulation in response to cytokines. Pirfenidone, which has demonstrated activity in multiple fibrotic indications, is currently in Phase II clinical development for fibrotic diseases of the lung, kidney and liver.

### Interferon Gamma (IFN-γ)

IFN-γ is a naturally glycoprotein cytokine, acting synergistically with other cytokines to exert its wide ranging effects. IFN-γ is a naturally occurring glycoprotein having a molecular weight of about 17.000 which can be commercially produced today also by recombinant techniques.

For improvement of the pharmacokinetic features: IFN-γ can be modified by pegylation to obtain PEG-IFN-γ. In addition to injection techniques, IFN-γ can be administered by inhalation to the lungs to diminish undesirable side effects. IFN-γ exhibits antiviral activity and together with other cytokines it plays an pivotal immunoregulatory role within the human immune system (an immunostimulating or an immunosuppressive effect, dependent on cell activation and location). Effects of IFN-γ include the induction of MHC class II antigens, macrophage activation, increased immunoglobulin production from B lymphocytes and enhanced NK cell activity.

The general antifibrotic properties of IFN-γ are well-documented (Lortat-Jacob H, Kleinman H K, Grimaud J A. High-affinity binding of interferon-gamma to a basement membrane complex (matrigel). J Clin Invest. 1991; 87: 878-83.; Narayanan A S, Whithey J, Souza A, Raghu G. Effect of gamma-interferon on collagen synthesis by normal and fibrotic human lung fibroblasts. Chest. 1992 May; 101 (5):1326-31; Hjelmeland L M, Li J W, Toth C A, Landers M B 3d. Antifibrotic and uveito-genic properties of gamma interferon in the rabbit eye. Graefes Arch Clin Exp Opthalmol. 1992; 230(1):84-90; Hyde D M, Henderson T S, Giri S N, Tyler N K, Stovall M Y. Effect of murine gamma interferon on the cellular responses to bleomycin in mice. Exp Lung Res. 1988; 14(5):687-704.). It has already been demonstrated that IFN-γ causes a reduced expression of TGF-β₁ together with a reduction in the amount of fibrosis (Giri S N, Hyde D M, Marafino B J Jr. Ameliorating effect of murine interferon gamma on bleomycin-induced lung collagen fibrosis in mice. Biochem Med Metab Biol. 1986 October; 36(2):194-7.). When using IFN-γ under clinical conditions, the transcription of TGF-β1 and that of CTGF are significantly diminished after six months of therapy (Ulloa L, Doody J, Massague J. Inhibition of transforming growth factor-beta/SMAD signalling by the interferon-gamma/STAT pathway. Nature 1999; 397: 710-3.). The simultaneous improvement of lung function in individuals suffering from IPF receiving IFN-γ provides additional support for the hypothesis that the mesenchymal activation in individuals with lung fibrosis depends, at least in part, on the continuous overexpression of TGF-β₁ and CTGF (Ziesche R, Hofbauer E, Wittmann K, Petkov V, Block L H. A preliminary study of long-term treatment with interferon gamma-1b and low-dose prednisolone in individuals with idiopathic pulmonary fibrosis. N Engl J. Med. 1999; 341: 1264-9; EP0795332A2.). Moreover, these results suggest that an acquired deficiency of IFN-γ may be one reason for the exaggerated wound healing process characteristic of progressive organ fibrosis.

IFN-γ was shown to be the first clinically applicable antifibrotic drug. As any endogenous substance, IFN-γ has pleiotropic effects dependent on the existing conditions within the body. In case of an exaggerated, yet non- or low-inflammatory wound healing, such as in IPF, the drug exerts powerful highly beneficial anti-fibrotic properties. However, in the presence of accompanying infections, such as chronic bacterial, viral or fungal infections, the drug will add to the inflammatory process and even reinforce, by intensifying tissue repair, the fibrotic process.

Thus, for a safe and effective use of the drug, it is necessary to molecularly measure the state of tissue response prior to application of the drug. The absence of the mRNA transcription of the gene for IFN-γ in the presence of intensified transcription of factors of wound healing may serve as the first vague discriminator between fibrosis as a result of deregulated wound healing itself, versus fibrosis as a result of intensified inflammation. The first attempts to analyze gene expression patterns in pulmonary fibrosis in animal models demonstrate the power of this tool to discriminate between the fibrotic process itself and accompanying mechanisms (Kaminski et al., Proc Natl Acad Sci USA 2000 Feb. 15; 97(4):1778-83 Global analysis of gene expression in pulmonary fibrosis reveals distinct programs regulating lung inflammation and fibrosis; Katsuma et al., Biochem Biophys Res Commun 2001 Nov. 9; 288(4):747-51 Molecular monitoring of bleomycin-induced pulmonary fibrosis by cDNA microarray-based gene expression profiling). A medication which combines the molecular diagnosis (transcription analysis in diseased ILD patients compared to control populations—healthy individuals or patients suffering from different lung diseases—with a distinct quantitative, specific expression difference to those) with proven effectiveness of a antifibrotic drug like IFN-γ or pirfenidone under clinical conditions provides the essential step for the first bio-medical based treatment of these multifunctional diseases.

To sum up, the real effectiveness of IFN-γ therapy mainly depends on four conditions:
(a) the proof of IFN-γ deficiency in pulmonary tissue;
(b) the control of infections or other inflammatory conditions, either pre-existing or acquired during therapy;
(c) the gene expression profiling of the patients suspected to have an interstitial lung disease;
(d) the duration of disease-related symptoms, reflecting the extent of completed scarring.

Clinical experience suggests that repeated phases of increased inflammation due to a large variety of infectious agents are the most common reason for a failure of IFN-γ in the treatment of ILD. Thus, clinical and molecular control of inflammation and infections, especially in the peripheral bronchi is crucial for a success of an anti-fibrotic treatment with IFN-γ.

Thus it is goal of the work of the present invention to improve the treatment of lung diseases, especially ILD and its various subforms with novel pharmaceuticals using either pirfenidone or IFN-γ in pharmaceutical composition with a diagnostic array of candidate polynucleotides (genechip).

### Disease-Related Gene Expression Profiles

With the completion of the Human Genome Project sequencing, biomedical research will be revolutionised by the ability to carry out investigations on a genome wide scale:
Arch Opthalmol 2001 November; 119(11):1629-34
Microarray analysis of gene expression in human donor corneas.
Jun A S et al.
Ann Surg 2001 December; 234(6):769-78; discussion 778-9
Identification of disease-specific genes in chronic pancreatitis using DNA array technology. Friess H et al.
DNA Cell Biol 2001 November; 20(11):683-95
A gene expression profile of Alzheimer's disease.
Loring J F et al.
Pharmacogenomics J 2001; 1(4):272-87
Molecular classification of psoriasis disease-associated genes through pharmacogenomic expression profiling.
Oestereicher J L et al.

Gene expression technology is gaining increasingly widespread use as a means to determine the expression of potentially all human genes at the level of messenger RNA. Gene specific sequences (oligo-, polynucleotides or cDNAs) are immobilised on arrays, hybridised with complex probes represented by a mixture of cDNAs of respective samples from human biopsies and other human materials (reversely transcribed from messenger RNA), and labelled with different dyes. Hybridised slides are analysed with sensitive scanning methodologies. Genechips containing gene sequences deliver fast and excellent overview of the expression pattern of the biological samples. Thus, the genome-wide gene expression analysis provides new insights into causes of disease, and elucidates the as yet unknown biological gene expression ****variations between seemingly similar diseases in defined detection ranges, leading to a new classification system valuable in accurate diagnosis.

Important applications include:
(a) development of a more global understanding of the qualitative and quantitative gene expression profiles that contribute to disease processes;
(b) discovery of new diagnostic and prognostic indicators and biomarkers of therapeutic response;
(c) identification and validation of new molecular targets for drug development;
(d) provision of an improved understanding of the molecular mode of action during lead identification and optimisation;
(e) designing more specific and efficient treatment strategies;
(f) prediction of potential side-effects during development and toxicology studies;
(g) confirmation of molecular modes of action during hypothesis-testing clinical trials;
(h) identification of genes involved in conferring drug sensitivity and resistance;
(i) prediction of patients most likely to benefit from the drug and use in general pharmacogenomic studies.

As a result of further technological improvements, array-based genechips will become essential part for clinical interventions in identifying and quantifying individual gene-expression patterns for purposes of patient-oriented therapy, and in establishing a method for predicting efficacy of drugs for individual patients, given the functional complexity of most diseases, especially those involving fibroproliferative processes. Considering the fact that inflammatory diseases reflect qualitative and quantitative changes in the activity of certain genes and/or gene clusters, the molecular assessment will allow for a higher specificity and efficacy of medical treatments and will be considered integral part of the therapeutic composition.

To achieve this goal, new molecular markers and markers of progression are needed for improved staging and for better assessment of diagnosis and treatment of complex diseases.

Gene expression profiling techniques in combination with therapeutics offer the opportunity to discover such pathophysiologically relevant markers of disease.

In the context of this disclosure, a number of terms shall be utilized.

The term “polynucleotide” refers to a polymer of RNA or DNA that is single-stranded, optionally containing synthetic, non-natural or altered nucleotide bases. A polynucleotide in the form of a polymer of DNA may be comprised of one or more segments of cDNA, genomic DNA or synthetic DNA.

The term “subsequence” refers to a sequence of nucleic acids that comprises a part of a longer sequence of nucleic acids. The term “immobilized on a support” means bound directly or indirectly thereto including attachment by covalent binding, hydrogen bonding, ionic interaction, hydrophobic interaction or otherwise.

An aspect of the invention relates to polynucleotide arrays, which allows to qualitatively and quantitatively study mRNA expression levels of selected candidate genes in human materials.

Polynucleotide or DNA arrays consist of large numbers of DNA molecules spotted in a systematic order on a solid support or substrate such as a nylon membrane, glass slide, glass beads or a silicon chip. Depending on the size of each DNA spot on the array, DNA arrays can be categorized as microarrays (each DNA spot has a diameter less than 250 microns) and macroarrays (spot diameter is grater than 300 microns). When the solid substrate used is small in size, arrays are also referred to as DNA chips. Depending on the spotting technique used, the number of spots on a glass microarray can range from hundreds to tens of thousands.

DNA microarrays serve a variety of purposes, including gene expression profiling, de novo gene sequencing, gene mutation analysis, gene mapping and genotyping. cDNA microarrays are printed with distinct cDNA clones isolated from cDNA libraries. Therefore, each spot represents an expressed gene, since it is derived from a distinct mRNA.

Typically, a method of monitoring gene expression involves providing
(1) a pool of sample polynucleotides comprising RNA transcripts of target genes or nucleic acids derived from the RNA transcripts;
(2) hybridizing the sample polynucleotides or nucleic acids derived from the RNA transcripts to an array of probes (for example, polynucleotides obtained from a polynucleotide library including control probes, and
(3) detecting the hybridized double-stranded polynucleotides/nucleic acids. The label used to mark polynucleotide samples is selected from the group consisting of radioactive, calorimetric, enzymatic, molecular amplification, bioluminescent or fluorescent label.

The invention relates also to any polynucleotide library as previously described wherein said polynucleotides are immobilized on a solid support in order to form a polynucleotide array.

Preferably the support is selected from the group consisting of a nylon membrane, glass slide, glass beads, or a silicon chip.

The invention relates to a polynucleotide library useful in the molecular characterization of a fibrotic interstitial lung disease like Idiopathic Pulmonary Fibrosis (IPF), the library including a pool of polynucleotide sequences or subsequences thereof wherein the sequences or subsequences are either underexpressed or overexpressed in IPF diseased cells. Preferably, a set of sequences is selected from oligo- or polynucleotide probes having a sequence defined by, or correlated to, or derived from the group of genes consisting of candidate genes indicated in the following list, representing increased gene expression levels of IPF cells versus cells from patients with a different lung disorder:

PTH-responsive osteosarcoma B1 protein, AF095771.1
matrix associated, actin dependent regulator of chromatin, subfamily f, member 1, AF231056.1 deleted in lung and esophageal cancer 1 (DLEC1), NM_{—}007337.1
major histocompatibility complex, class II, DQ beta 1, AW276186
SB class II histocompatibility antigen alpha-chain, AI128225 mucin 4, tracheobronchial, AJ242547.1
forkhead box J1 (FOXJ1), U69537.1
hypothetical protein FLJ21616, NM_{—}024567.1
neuronal specific transcription factor DAT1, AF258348.1
hematopoietic PBX-interacting protein, BF344265
proline oxidase homolog, AA074145
mucin 5, subtype B, tracheobronchial, AI697108
golgi membrane protein GP73, AF236056.1
ATP citrate lyase, U18197.1
NG22 protein, NM_{—}025257.1
cDNA DKFZp434A2322, AL137706.1
hepatocyte nuclear factor 3, alpha, U39840.1
major histocompatibility complex, class II, DQ alpha 1, X00452.1
myosin regulatory light chain 2, smooth muscle isoform, J02854.1
plexin B1, AV693216
pyruvate kinase, muscle, BC000481.1
tetraspanin TM4-C, AF133425.1
insulin-like growth factor binding protein 2 (36 kD), BC004312.1
FLJ13945 fis, clone Y79AA1000969, AU160041
hypothetical protein DKFZp586M1120, NM_{—}031294.1
CD24 signal transducer, L33930
hypothetical protein FLJ23571, NM_{—}025111.1
glutathione S-transferase M2 (muscle), M63509.1
cadherin 1, type 1, E-cadherin (epithelial), L08599.1
NTT5 protein, AF265578.1
lipocalin 2 (oncogene 24p3), NM_{—}005564.1
myotonic dystrophy kinase (DM kinase), L08835
uncoupling protein 2 (mitochondrial, proton carrier), U76367.1
dynein intermediate chain 2, NM_{—}023036.1
discoidin receptor tyrosine kinase isoform b, discoidin domain receptor family, member 1, NM_{—}001954.2
sperm associated antigen 6, AF079363.1
hypothetical protein FLJ23049, NM_{—}024687.1
nasopharyngeal epithelium specific protein 1, AF094758.1
nuclear receptor subfamily 4, group A, member 2, NM_{—}006186.1
hypothetical protein FLJ22215, BC003543.1
non-specific cross reacting antigen, M18728.1
amylase, alpha 1A; salivary (AMY1A), NM_{—}004038.1
carcinoembryonic antigen-related cell adhesion molecule 6 (non-specific cross reacting antigen), BC005008.1
glutathione S-transferase subunit 4 (EC 2.5.1.18), X08020.1
SH3-containing protein SH3GLB2, AF257319.1
KDEL (Lys-Asp-Glu-Leu) endoplasmic reticulum protein retention receptor 1, NM_{—}006801.1
anterior gradient 2 (Xenepus laevis) homolog, AF038451.1
sv7-MUC4 apomucin, mucin 4, tracheobronchial, AJ242547.1
stratifin, BC000329.1
connective tissue growth factor, M92934.1
cytochrome P450-IIB (hIIB3), M29873.1
filamin A, alpha (actin-binding protein-280), AW051856
membrane glycoprotein LIG-1, AB050468.1
E74-like factor 3 (ets domain transcription factor, epithelial-specific), U73844.1
elastin (supravalvular aortic stenosis, Williams-Beuren syndrome), M36860.1
ephrin receptor EPHA3, AF213459.1
fibrillin 1 (Marfan syndrome), L113923.1
discs, large (Drosophila) homolog 1, U13896.1
lysyl oxidase-like 1 (LOXL1), L21186.1
calumenin, U67280.1
male germ cell-associated kinase, NM_{—}005906.2
plectin 1, intermediate filament binding protein, 500 kD, Z54367
peroxisome biogenesis factor 1, AF026086.1
mast cell tryptase beta III, AF099143
ataxia-telangiectasia group D-associated protein, AF230388.1
hypothetical protein FLJ10921, NM_{—}018272.1
biglycan, BC002416.1
BLu protein, AC002481
CGI-92 protein, AF 151850.1
adaptor-related protein complex 1, mu 2 subunit, B0003387.1
keratin 15, BC002641.1
B7 protein, U72508.1
S100 calcium-binding protein A2, BC002829.1
MUF1 protein, BC004953.1
cholesterol 25-hydroxylase, AF059214.1
cytidine monophosphate-N-acetylneuraminic acid hydroxylase (CMP-N-acetylneuraminate monooxygenase), AF074480.1
neuropilin 2, AF022859.1
Fas-interacting serinethreonine kinase 3, homeodomaininteracting protein kinase 3, AF305239.1
a disintegrin and metalloproteinase domain 28 (ADAM28), transcript variant 2, AF137334.1
cDNA DKFZp434A119, AW663632
complement component 6, J05064.1
cytokeratin 17, Z19574
wingless-type MMTV integration site family, member 5A, AI968085
matrix metalloproteinase 7 (matrilysin, uterine), BC003635.1
leiomodin 1 (smooth muscle), NM_{—}012134.1
Cip1-interacting zinc finger protein, AB030835.1
cyclin-dependent kinase inhibitor IA (p21, Cip 1), BC000275.1
integrin, alpha 7, AF032108.1
DKFZP586G011 protein, BG289527
fatty acid binding protein 6, ileal (gastrotropin), U19869.1
glutathione S-transferase M4, M96234.1
Ras-related associated with diabetes, L24564.1
claudin 3, AB000714.1
matrix metalloproteinase 10 (stromelysin 2), BC002591.1
fibulin 2, NM_{—}001998.1
serine threonine kinase 11 (STK11), AF035625
eukaryotic translation initiation factor 1A: AF000987.1
DEADH (Asp-Glu-Ala-AspHis) box polypeptide: AF000985.1
ribosomal protein S4: AF116711.1
ubiquitin specific protease 9: AF000986.2
SMC (mouse) homolog: U52191.1
myelin basic protein: LI 8865.1
S100 calcium-binding protein: NM_{—}005980.1

### Jagged2 (JAG2): AF003521.1

latent transforming growth factor beta binding protein 4: NM_{—}003573.1
microtubule-associated protein, RPEB family, member 3: AB025186.1
Unknown (protein for MGC:2854): BC003629.1
clone=IMAGE-2406340: AI830563
AQP3 gene for aquaporine 3 (water channel): AB001325
cDNA DKFZp434A119
fenestrated-endothelial linked structure protein (FELS), PV1 protein (PLVAP): AF326591.1
LUNX protein; PLUNC (palate lung and nasal epithelium clone); tracheal epithelium enriched protein (LOC51297): AB024937.1
RAB, member of RAS oncogene family-like 2A: AF095350.1
chromosome 11 open reading frame 16: NM_{—}020643.1
hypothetical protein FLJ23049: NM_{—}024687.1
hypothetical protein FLJ 1767: NM_{—}024593.1
dynein, axonemal, intermediate polypeptide: AF091619.1
brain specific protein (LOC51673): AF132972.1
MUC4 apomucin, mucin 4, tracheobronchial: AJ242547.1
myotonin protein kinase (DM): M87313.1
cytokeratin 4: X07695.1
KIAA0362 gene, MCF.2 cell line derived transforming sequence-like: AB002360.1
cytokeratin 17: Z19574
LIM domain protein: BC003096.1
E74-like factor 3 (ets domain transcription factor, epithelial-specific: U73844.1
fatty acid binding protein 6, ileal (gastrotropin): U19869.1
sperm associated antigen 6: AF079363.1
eyes absent (Drosophila) homolog 2: U71207.1
phosphatidic acid phosphatase type 2C: BC002806.1
epoxide hydrolase 2, cytoplasmic: AF233334.1
tubulin, beta, 2: BC002783.1
heat shock 105 kD: D86956.1
villin 2 (ezrin): J05021.1
a disintegrin and metalloproteinase domain 28 (ADAM28), transcript variant 3: AF137335.1
deleted in lung and esophageal cancer 1: NM_{—}007337.1
arachidonate 15-lipoxygenase: NM_{—}001140.1
UDP glycosyltransferase 1 family, polypeptide A1: M57899.1
hypothetical protein PRO2834: AF119903.1
lectin, galactoside-binding, soluble, 7 (galectin 7): L07769.1
B7 protein: U72508.1
ephrin receptor EPHA3: AF213459.1
forkhead box J1: U69537.1
BLu protein: U70824.1
aldehyde dehydrogenase 3 family, member A1: BC004370.1
NG22 protein: NM_{—}025257.1
small inducible cytokine subfamily A (Cys-Cys), member 14: NM_{—}004166.1
cysteine-rich protein 1 (intestinal): BC002738.1
putative GTP-binding protein similar to RAYRABIC: BC000566.1
integrin, beta 4: NM_{—}000213.1
serine (or cysteine) proteinase inhibitor, clade B (ovalbumin), member 5 (SERPINB5): U04313.1
Ras-related associated with diabetes: L24564.1
hepatic leukemia factor: M95585.1
keratin 15: BC002641.1
nuclear receptor subfamily 4, group A, member 2: NM_{—}006186.1
sialyltransferase: U14550.1
glutathione S-transferase M2 (muscle): M63509.1
hypothetical protein FLJ13110: NM_{—}022912.1
S100 calcium-binding protein A2: NM_{—}005978.2
collagen, type VII, alpha 1 (epidermolysis bullosa, dystrophic, dominant and recessive) L02870.1
claudin 3: AB000714.1
insulin-like growth factor binding protein 6: BC003507.1
fibroblast growth factor receptor 2 (bacteria-expressed kinase, keratinocyte growth factor receptor, craniofacial dysostosis 1, Crouzon syndrome, Pfeiffer syndrome, Jackson-Weiss
syndrome): M80634.1
insulin-like growth factor 1 receptor: NM_{—}000875.2
insulin-like growth factor binding protein 2 (36 kD): M35410.1
ataxia-telangiectasia group D-associated protein: AF230388.1
keratin 5 (epidermolysis bullosa simplex, Dowling-MearaKobnerWeber-Cockayne types): M21389.1
Duffy blood group: U01839.1
transforming growth factor β1: NM_{—}000660
wherein each oligo- or polynucleotide probe is obtainable on the basis of structural information mediated by the sequence data provided by the respective accession number set out for each candidate gene.

Yet another set of sequences is selected from oligo- or polynucleotide probes having a sequence defined by, or correlated to, or derived from the group of genes consisting of candidate genes indicated in the following list, representing decreased gene expression levels of IPF cells versus cells from patients with different lung disorder:

sorting nexin 10 (SNX10), AF 121860.1
phospholipase A2, group IIA (platelets, synovial fluid), M22430.1
hemoglobin alpha-1 globin chain (HBA1), AF349571.1
macrophage scavenger receptor 1, AI299239
surfactant, pulmonary-associated protein C, BC005913.1
disintegrin protease (M12.219), NM_{—}014479.1 retinol-binding protein 4, interstitial, AF119868.1
major histocompatibility complex, class II, DR beta 3, M27635.1
adipose differentiation-related protein, BC005127.1
hemoglobin, delta, NM_{—}000519.2
beta-2-microglobulin, AW188940
hemoglobin, alpha 2, BC005931.1
protein C receptor, endothelial (EPCR), L35545.1
thymosin, beta 10, M92381.1 apolipoprotein C-I, W79394
hypothetical protein, AL133067.1
proteoglycan 4, (megakaryocyte stimulating factor, articular superficial zone protein), U70136.1
tetranectin (plasminogen-binding protein), NM_{—}003278.1
platelet factor 4, M25897.1
tissue factor pathway inhibitor 2, BC005330.1
fatty acid binding protein 4, adipocyte, BC003672.1
cathepsin B (CTSB), M14221.1
transmembrane 4 superfamily member 1, M90657.1
MHC class I HLA-B51 major histocompatibility complex, class I, E, M31183.1
endothelial PAS domain protein 1, U51626.1
Apo-2 ligand tumor necrosis factor (ligand) superfamily, member 10, U37518.1
haptoglobin-related protein, NM_{—}020995.1
Rho guanine exchange factor (GEF) 12, AF119898.1
major histocompatibility complex, class II, DR beta 5, M11867.1
interferon, gamma-inducible protein 30, AF097362.1
ferritin, light polypeptide, BG537190
prosaposin (variant Gaucher disease and variant metachromatic leukodystrophy), BC004275.1
calcium-binding protein A4 (calvasculin, metastasin,), NM_{—}002961.2
hemoglobin, beta, M25079.1
CDW52 antigen (CAMPATH-1 antigen), BC000644.1
aldehyde dehydrogenase 1 family, member A2, AB015226.1
cathepsin Z, AF032906.1
MHC HLA-B39 major histocompatibility complex, class I, B, L37880.1
major histocompatibility complex, class II, DQ alpha 1, M33906.1
fibroblast growth factor 9 (glia-activating factor), D14838.1 hemoglobin, alpha 2, AF097635.1
transferrin receptor (p90, CD71), BC001188.1
complement component 3 (C3), K02765.1
cDNA DKFZp564D166, AL050025.1
complement component 1, q subcomponent, beta polypeptide, NM_{—}000491.2
small inducible cytokine subfamily A (Cys-Cys), member 18, pulmonary and activation-regulated, AB000221.1
reticulon 1, L10333.1
major histocompatibility complex, class II, DR beta 1, M33600.1
haptoglobin, L29394.1
acid phosphatase 5, tartrate resistant, J04430.1
cytochrome P450, subfamily XXVIIA (steroid 27-hydroxylase, cerebrotendinous xanthomatosis), polypeptide 1, M62401.1
CD36 antigen (collagen type I receptor, thrombospondin receptor), M24795.1
calbindin 2, (29 kD, calretinin), NM_{—}001740.2
alpha-2-HS-glycoprotein, BG538564
cDNA DKFZp564A132, AL049963.1
fibronectin 1, AF130095.1
phosphodiesterase 4C, cAMP-specific, NM_{—}000923.1
transcription factor 7 (T-cell specific, HMG-box), NM_{—}003202.1
found in inflammatory zone 3 (FIZZ3), AF323081.1
claudin 15, NM_{—}014343.1
carboxypeptidase B1 (tissue), M81057.1
hypothetical protein FLJ 14054, NM_{—}024563.1
bone marrow stromal cell antigen 1, D21878.120
interleukin 7 receptor, M29696.1
procollagen C-endopeptidase enhancer 2, AF098269.1
calcium-binding protein A8 (calgranulin A), AW238654
cDNA DKFZp564D193, AL049252.1
major histocompatibility complex, class II, DP beta 1, J03041.1
human leukocyte antigen C alpha chain, major histocompatibility complex, class I, C,
BCM-like membrane protein precursor, AF144235.1
CD14 antigen, M86511.1
pulmonary surfactant protein (SP5), J03553
signal transducer and activator of transcription 1, 91 kD, BC002704.1
Wilms tumor 1 (WT1), transcript variant D, NM_{—}024424.1
annexin A8, BC004376.1
macrophage receptor with collagenous structure, MARC0, AF035819.1
surfactant, pulmonary-associated protein A2, NM_{—}006926.1
solute carrier family 6 (neurotransmitter transporter, serotonin), member 4, L05568.1
chitinase 1 (chitotriosidase), U29615.1
lung type-I cell membrane-associated glycoprotein, AU154455
fibronectin leucine rich transmembrane protein 2, AB007865.1
gamma-aminobutyric acid (GABA) A receptor, alpha 5, BF966183
hypothetical protein FLJ12983, NM_{—}024856.1
sialophorin (gpL115, leukosialin, CD43), J04536.1
cerebellar degeneration-related protein (34 kD), M16965.1
hydroxyacid oxidase 2 (long chain), hydroxy-delta-5-steroid dehydrogenase (3 beta- and steroid
delta-isomerase 2), AL359553
CLONE=IMAGE: 1032795=Hs.83623 nuclear receptor subfamily 1, group I, member 3
small inducible cytokine subfamily C, member 2: NM_{—}003175.1
hypothetical protein similar to swine acylneuraminate lyase: NM_{—}030769.1
fibrinogen, gamma polypeptide: AF118092.1
thrombospondin 1: NM_{—}003246.1
SAM domain, SH3 domain and nuclear localisation signals, 1: AF222927.1
chitinase 3-like 1 (cartilage glycoprotein-39): M80927.1
cathepsin Z: AF032906.1
CLONE=IMAGE:3579023 collagen, type XIV, alpha 1 (undulin)
chloride intracellular channel 2: NM_{—}001289.2
monokine induced by gamma interferon: NM_{—}002416.1
KIAA0433 protein: NM_{—}015216.1
tumor necrosis factor, alpha-induced protein 6: NM_{—}007115.125
signal transducer and activator of transcription 1, 91 kD: BC002704.1
thrombospondin 2: L12350.1
collagen, type IV, alpha 3 (Goodpasture antigen): NM_{—}000091.1
integrin, beta-like 1 (with EGF-like repeat domains): AF072752.1
diubiquitin: NM_{—}006398.1
protease, cysteine, 1 (legumain): D55696.1
cytochrome P450, subfamily I (dioxin-inducible), polypeptide 1 (glaucoma 3, primary infantile): U03688.1
integrin, alpha 1: X68742.1
GABA-B receptor, G protein-coupled receptor 51: AF056085.1
KIAA1199 protein: AB033025.1
collagen, type V, alpha 2: NM_{—}000393.1
interleukin 13 receptor, alpha 2: U70981.1
translocase of inner mitochondrial membrane 8 (yeast) homolog A: BC005236.1
steroid sulfatase (microsomal), arylsulfatase C, isozyme S: M16505.1
CLONE=IMAGE:1982571 ATPase, H+ transporting, lysosomal (vacuolar proton pump) 9 kD
proteoglycan 4, (megakaryocyte stimulating factor, articular superficial zone protein): U70136.1
nidogen (enactin): M30269.1
KIAA1598 protein: AU157109
vascular cell adhesion molecule 1: M60335.1
guanylate binding protein 1, interferon-inducible, 67 kD: BC002666.1
cDNA DKFZp586E1124
apolipoprotein H (beta-2-glycoprotein I): M62839.1
ribosomal protein L37a: BE857772
cDNA DKFZp564A132
cathepsin S: M86553.1
a disintegrin and metalloproteinase domain 9 (meltrin gamma) (ADAM9): U41766.1
zinc finger protein 331: AF272148.1
lysosomal-associated membrane protein 2: J04183.1
carboxypeptidase M: NM_{—}001874.1
collagen, type I, alpha 2: NM_{—}000089.1
P311 protein: U36189.1
KIAA0372 gene product: AB002370.1
Human T cell-specific protein RANTES: M21121
interferon-gamma-inducible indoleamine 2,3-dioxygenase (IDO): M34455.1
hypothetical protein FLJ10430: NM_{—}018092.1
transcription factor ISGF-3: M97935
interleukin 1 receptor-like 1 (IL1RL1): NM_{—}003856.1
putative alpha chemokine (H174), small inducible cytokine subfamily B (Cys-X-Cys), member 11: AF002985.1
small inducible cytokine subfamily B (Cys-X-Cys), member 10: NM_{—}001565.1
mesoderm specific transcript (mouse) homolog: BC002413.1
carboxypeptidase B-like protein: AB011969.1
CD2 antigen (p50), sheep red blood cell receptor: M16445.
interferon, alpha-inducible protein (clone IFI-6-16): NM_{—}022872.1
RAS guanyl releasing protein 1 (calcium. and DAG-regulated): AF081195.1
lipase, endothelial: AF118767.1
fatty-acid-Coenzyme A ligase, long-chain 4: NM_{—}022977.1
klotho: AB005142.1
chondroitin sulfate proteoglycan 2 (versican): NM_{—}004385.1
hypothetical protein, expressed in osteoblast: AB000115.1
CD14 antigen: M86511.1
CGI-83 protein: BC000878.1
leucine aminopeptidase: AF061738.1
UDP-Gal:betaGlcNAc beta 1,3-galactosyltransferase, polypeptide 3: AB050855.1
protocadherin alpha 12: AF152308.1
neuroglycan C: AF059274
neuroligin: AI338338
HSPC156 protein: AF161505.1
hypothetical protein FLJ13310: NM_{—}025118.1
eosinophil chemotactic cytokine (TSA1902): AB025008.1
aminopeptidase: AF191545.1
semaphorin sem2: AB029496.1
protocadherin 12: AF231025.1
X transporter protein 3: NM_{—}020208.1
transmembrane 4 superfamily member (tetraspan NET-2): AF124522.1
hypothetical protein FLJ 10970: NM_{—}018286.1
perforin 1 (pore forming protein): M28393.1
natural killer cell group 7 sequence: NM_{—}005601.1
hypothetical protein DKFZp761N09121: BF435376
integrin, alpha 4 (antigen CD49D, alpha 4 subunit of VLA-4 receptor): BG532690 chitinase 3-like 2: U58515.1
FYN oncogene: N20923
relaxin 1 (H1): BC005956.1
hydroxyprostaglandin dehydrogenase 15-(NAD): U63296.1
sulfotransferase family, cytosolic, 1C, member 1: AF186254.1
TGF-b superfamily receptor type I: L17075.1 granzyme B (granzyme 2, cytotoxic T-lymphocyte-associated serine esterase 1): M36118.1
cystatin F (leukocystatin): AF031824.1
regulator of G protein signaling-Z (RGSZ1): AF060877.2
clone MGC:12387: M16942.1
zinc-alpha2-glycoprotein: D90427.1
BCG induced integral membrane protein BIGMo-103: AB040120.1
nectin-like protein 2 (NECL2): AF132811.1
CD209 antigen-like: AB015629.1
solute carrier family 6 (neurotransmitter transporter, serotonin), member 4: L05568.1
MAD (mothers against decapentaplegic, Drosophila) homolog 6: U59914.1
latrophilin: AF 104939.1
platelet factor 4: M25897.1
calcitonin receptor-like: L76380.1
matrilin 3: NM_{—}002381.2
solute carrier family 14 (urea transporter), member 1 (Kidd blood group): U35735.1
interleukin 7 receptor: M29696.1
MAP kinase kinase 6 (MKK6), mitogen-activated protein kinase kinase 6: U39656.1
protocadherin 17: AF029343.1
granulysin: NM_{—}006433.2
interferon-stimulated protein, 15 kDa (ISG15): M13755.1
cadherin 5, type 2, VE-cadherin (vascularepithelium): U84722.1
thrombomodulin: M16552.1
interferon, alpha-inducible protein 27: NM_{—}005532.1
interferon-gamma: X87308
wherein each oligo- or polynucleotide probe is obtainable on the basis of structural information mediated by the sequence data provided by the respective accession number set out for each candidate gene.

For example, suitable sequences which may be used for oligonucleotide design may be downloaded from the NCBI GenBank (http://www.ncbi.nlm.gov/GenBank/index.html) using the Accession number listed behind the gene name.

All accession numbers give access to mRNA sequences; since the cDNA of the patients is reverse transcribed, the oligonucleotide have to sense strand and are therefore identical with the mRNA sequence. Preferably the pool of polynucleotide sequences or subsequences correspond substantially to the polynucleotide sequences useful in qualitative and quantitative differentiating a normal cell, or a cell from a patient suffering from non-fibrosing lung disease, from a cell of a patient with interstitial lung disease, most preferably with Idiopathic Pulmonary Fibrosis (IPF).

The invention concerns the part of pharmaceutical composition for detecting differentially expressed polynucleotide sequences which are correlated with IPF, said part comprising: a) obtaining a polynucleotide sample from a patient; and b) reacting the sample polynucleotide obtained in step (a) with a probe immobilized on a solid support wherein said probe comprises any of the polynucleotide sequences of the libraries previously described or an expression product encoded by any of the polynucleotide sequences of said libraries and c) detecting the reaction product of step (b) as a prerequisite for a subsequent administration of suitable drug.

The invention relates also to the part of pharmaceutical composition detecting differentially expressed polynucleotide sequences of the invention wherein the amount of reaction product of step (c) is compared to control samples.

The detection of differentially expressed polynucleotide sequences is used for detecting, diagnosing, staging, monitoring, prognosticating, preventing or treating conditions associated with ILD, and namely idiopathic pulmonary fibrosis IPF, hypersensivity pneumonitis, scleroderma, Systemic Lupus Erythematosus, Rheumatoid Arthritis, Churg-Strauss syndrome, Wegener's granulomatosis, and Goodpasture Syndrome.

The detection of differentially expressed polynucleotide sequences is particular useful wherein the product encoded by any of the polynucleotide sequences or subsequences is involved in a receptor-ligand reaction on which detection is based.

The invention relates also to detecting differentially expressed polynucleotide sequences previously described wherein the sample has been treated with the anti-ILD drug to be screened.

The invention also relates to a library of polynucleotides comprising a population of polynucleotide sequences overexpressed or underexpressed in cells derived from ILD patients. A particular embodiment of the invention relates to a polynucleotide library of corresponding substantially to any combination of at least one polynucleotide sequence selected among those included in each one of predefined polynucleotide sequences sets mentioned above.

The invention relates to polynucleotide libraries comprising at least one polynucleotide selected among those included in at least 50%, preferably 75% and more preferably 100% of said predefined sets, allowing to obtain a discriminating gene pattern, namely to distinguish between normal individuals and patients suffering from ILD, particularly from IPF.

Polynucleotide sequences library useful for the realization of the invention can comprise also any sequence comprised between 3′-end and 5′-end of each polynucleotide sequence sets as defined above, allowing the complete detection of the implicated genes.

The invention relates also to a polynucleotide library useful to differentiate a normal cell from a cell of ILD patients wherein the pool of polynucleotide sequences or subsequences correspond substantially to any combination of at least one polynucleotide sequence selected among those included in each one of predefined polynucleotide sequences sets indicated above, useful in differentiating a normal cell from a cell from ILD patients.

Differences in gene expression are accepted as different when the signals from patient material are at least two fold lower or higher than those from comparative material, be it from healthy volunteers material or from other diseased material. This threshold is commonly accepted for the evaluation of expression arrays.

The invention additionally provides a method for identifying gene expression or genomic DNA of infective agents including bacteria (Mycobacterium spec., Mycoplasma spec., Staphyllococcus aureus, treptococcus spec., Borrelia, Treponema pallidum, Leptospira interrogans, Campylobacter jejuni, fetus; Escherichia coli, EPEC, ETEC, EIEC, EHEC Salmonella enterica, Yersinia enterocolitica, Aeromonas spec., Campylobacter fetus, Moraxella catarrhalis, Moraxella catarrhalis, Brucella spec., Toxoplasma, Salmonella enterica, Shigella spec., Yersinia enterocolitica, Vibrio cholerae, Pseudomonas aeruginosa, Burkholderia cepacia, Stenotrophomonas maltophilia, Acinetobacter baumanii, Acitenobacter calcoaceticus, Klebsiella, Enterobacter, Citrobacter, Proteus, Serratia, Morganella, Providencia, Cardiobacterium hominis, Eikenella corrodens, Gardnerella vaginalis, Calymmatobacterium granulomatis, Bacteriodes, Porphyromonas, Prevotella, Fusobacterium, Rickettsia prowazekii, Bartonella bacilliformis, Bartonella henselae and Chlamydia spec.), yeasts, fungi, or viruses (retroviruses, adenoviruses, hepadnaviruses, herpesviruses, influenza viruses, paramyxoviruses) as cellular parasites in cells from patients with ILD.

### SUMMARY OF THE INVENTION

It was surprisingly found that a pharmaceutical composition of interferon gamma together with a gene expression analysis of the patients with lung diseases results in a faster and more successful treatment of lung diseases, preferably ILD and ILD related lung diseases. Thus, the invention relates to new pharmaceutical compositions and pharmaceutical kits comprising interferon gamma or pirfenidone and a disease-oriented gene expression analysis.

To sum up and in more detail, the invention relates to the following topics:
A pharmaceutical composition comprising
   (i) interferon gamma (IFN-γ), pegylated (PEG) IFN-γ), or pirfenidone, optionally together with a pharmaceutically acceptable carrier, diluent or excipient,
   (ii) a gene expression analysis of patients with interstitial lung disease (genechip);
a corresponding pharmaceutical composition, comprising additionally a glucocorticoid compound;
a corresponding pharmaceutical composition, wherein the medication is selected from the following group:
a pharmaceutical kit comprising
   (i) a package comprising IFN-γ, PEG-IFN-γ, or pirfenidone,
   (ii) a package comprising a gene expression analysis (genechip) of the patient with lung disease;
a corresponding pharmaceutical kit further comprising a package comprising a glucocorticoid compound;
a method or a use of a combination of a pharmaceutical composition or a pharmaceutical kit according as specified for the manufacture of a medicament for the treatment of interstitial lung diseases (ILD);
a corresponding method or use, wherein IFN-γ is administered to the individual in a single dose amount varying from 2.0 to 3.0 μg/kg body weight;
a corresponding method or use, wherein the glucocorticoid compound is administered to the individual in a single dose amount varying from 100 to 150 μg/kg body weight;
a corresponding method or use, wherein IFN-γ is administered to the individual by inhalation after a gene expression analysis.

### SHORT DESCRIPTION OF THE FIGURES

The foregoing and other features and aspects of the present invention will be best understood with reference to the following detailed description of a specific embodiment of the invention when read in conjunction with the accompanying drawings, wherein:

FIGS. 1A-1C depict the total lung capacity (TLC) (FIG. 1A), the partial pressure of arterial oxygen (PaO2) (FIG. 1B), and the forced vital capacity (FVC) of a patient with histologically proven UIP (open lung biopsy) (FIG. 1C) and failure of immunosuppressive treatment (f, 59 yr) treated with interferon gamma.

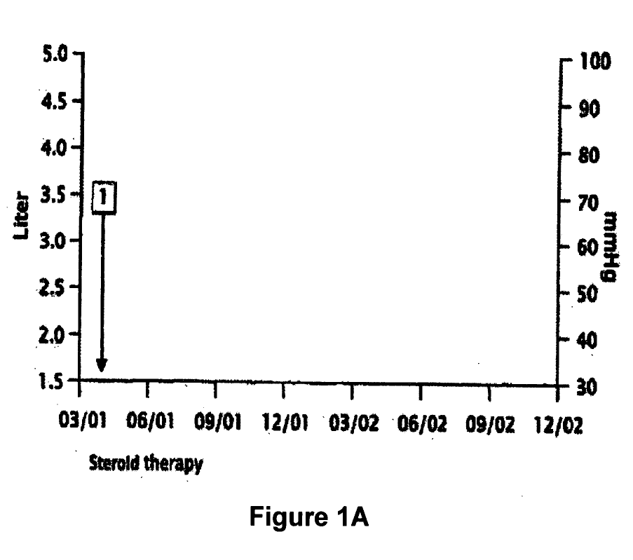

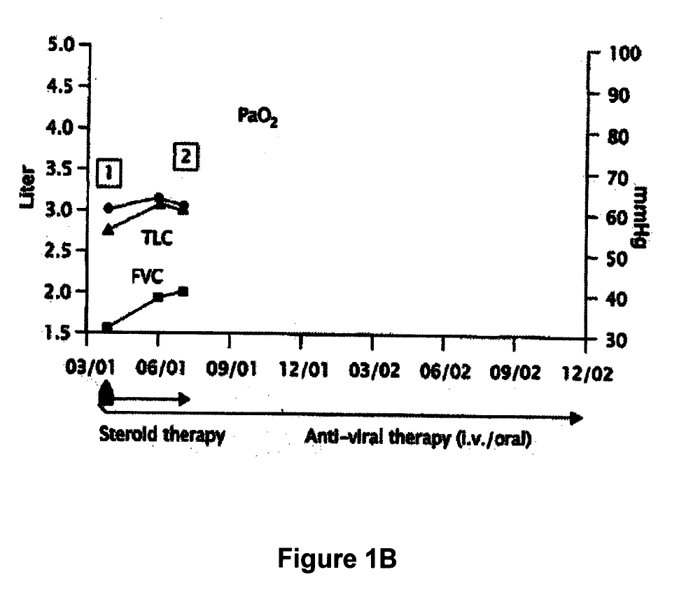

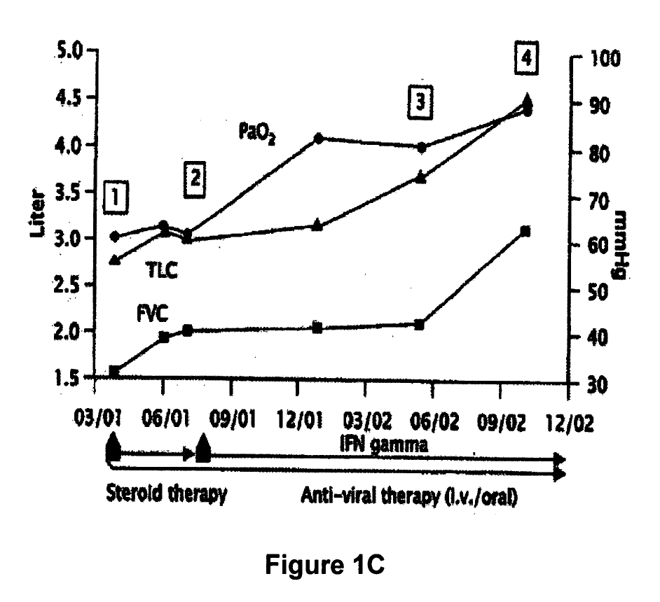

### DETAILED DESCRIPTION

Suitable compounds which have the therapeutic effect within the combination according to the invention, are, besides interferon gamma, pegylated interferon gamma, perfinidone, compounds which have the same, but also enhanced, biological activity of interferon gamma, pegylated interferon gamma, or pirfenidone in combination with the gene expression analysis of diseased patients.

The invention includes also derivatives, analogues, homologues, fusion proteins, stabilized forms, etc., of the disclosed drugs, as more specified above, which have the same biological activity as interferon gamma, or pirfenidone.

The term “same biological activity” means herein the same substantial biological, physiological or therapeutic activity or functionality, which however can be quantitatively enhanced or reduced compared with the relevant properties of said drugs.

The term “stabilized form” means a derivative or analogue wherein the parent drug was altered in order get more stability and increased half-life in blood and serum. Polypeptides and proteins may be protected against proteolysis by the attachment of chemical moieties. Such attachment may effectively block the proteolytic enzyme from physical contact with the protein backbone itself, and thus prevent degradation. Polyethylene glycol is one such chemical moiety which has been shown to protect against proteolysis (Sada, et al., J. Fermentation Bioengineering 71: 137-139, 1991). In addition to protection against proteolytic cleavage, chemical modification of biologically active proteins has been found to provide additional advantages under certain circumstances, such as increasing the stability and circulation time of the therapeutic protein and decreasing immunogenicity. (U.S. Pat. No. 4,179,337; Abuchowski et al., Enzymes as Drugs.; J. S. Holcerberg and J. Roberts, eds. pp. 367-383, 1981; Francis, Focus on Growth Factors 3: 4-10; EP 0 401 384). The addition of polyethylene glycol increases stability of the peptides and polypeptides of this invention at physiological pH as compared to non-pegylated compounds. The pegylated polypeptide/protein is also stabilized with regard to salts.

The term “fusion protein” means a compound, especially a stabilized form, consisting of a polypeptide according to the invention, preferably interferon gamma, which is fused to another peptide or protein.

The term “pharmaceutical kit” means a package comprising two or more packages or containers containing one or more, preferably one pharmaceutically active compound or agent and a gene expression analysis device, wherein the agent or compound in said packages or containers are administered to an individual after gene expression analysis is performed.

The pharmaceutical compositions according to the invention comprising interferon gamma or pirfenidone and a gene expression analysis as defined above and below can be used as medicament for the treatment of an individual.

The term “individual” preferably refers to mammals, especially humans.

The compound according to this invention, IFN-γ or pirfenidone, is used in a pharmaceutical formulations, comprising, as a rule, a pharmaceutically acceptable carrier, excipient or diluents. Techniques for the formulation and administration of the compounds of the present invention may be found in “Remington's Pharmaceutical Sciences” Mack Publishing Co., Easton Pa.

As used herein, the term “pharmaceutically acceptable carrier” means an inert, non toxic solid or liquid filler, diluent or encapsulating material, not reacting adversely with the active compound or with the individual, or any other formulation such as tablets, pills, dragees, capsules, gels, syrups, slurries, suspensions and the like. Suitable, preferably liquid carriers are well known in the art such as sterile water, saline, aqueous dextrose, sugar solutions, ethanol, glycols and oils, including those of petroleum, animal, vegetable, or synthetic origin, for example, peanut oil, soybean oil and mineral oil. Tablets and capsules for oral administration contain conventional excipients such as binding agents, fillers, diluents, tableting agents, lubricants, disintegrants, and wetting agents. The tablets may be coated according to methods well known in the art.

The formulations according to the invention may be administered as unit doses containing conventional non-toxic pharmaceutically acceptable carriers, diluents, adjuvants and vehicles which are typical for parenteral administration. The term “parenteral” includes herein subcutaneous, intravenous, intra-articular and intratracheal injection and infusion techniques. Parenteral compositions and combinations are most preferably administered intravenously either in a bolus form or as a constant fusion according to known procedures. Also other administrations such as oral administration or administration by inhalation or nasal spray are also object of the invention. Inhalation of vapors containing interferon gamma as specified is also a preferred way of administration. For inhalations the compound according to the invention is preferably brought in an aerosol form. Aerosols and techniques to make them are well known in the art. Aerosols applicable by inhalers containing a polypeptide of the invention, for example, interferon gamma are preferred if direct pulmonary symptoms have to be treated.

Unit doses according to the invention may contain daily required amounts of the compound according to the invention, or sub-multiples thereof to make up the desired dose. The optimum therapeutically acceptable dosage and dose rate for a given individual (mammals, including humans) depends on a variety of factors, such as the activity of the specific active material employed, the age, body weight, general health, sex, diet, time and route of administration, rate of clearance, enzyme activity, the object of the treatment, i.e., therapy or prophylaxis and the nature of the disease to be treated. Therefore, in the pharmaceutical compositions according to the invention for the therapy of an individual, a pharmaceutical effective daily dose of the respective compound in said composition is:

Interferon gamma (IFN-γ): It could be shown that interferon-γ is effective in the combination therapy according to the invention in a dose of 1.0-5.0 μg/kg body weight, preferably 2.0-3.0 μg/kg body weight, 1-5 times per week. The above-indicated single dosages of interferon-γ are administered parenteral, preferably subcutaneously to the patient. The doses of glucocorticoids which can be administered optionally together with IFN-γ to an individual vary according to the invention from 10-100 mg/single dose and more preferably from 15-80 mg, which corresponds to approximately 100-350 μg/kg body weight, preferably 100-150 μg/kg body weight.

1-32. (canceled)

33. A method of diagnosing Fibrotic Interstitial Lung Disease in an individual, comprising:
obtaining a pulmonary biological sample from said individual;
determining the expression level of interferon-gamma in said sample;
determining the expression levels of one or more genes that are either over expressed or under expressed in fibrotic interstitial lung disease; and
comparing the expression levels of said one or more genes and said interferon-gamma, with the expression level of the genes and expression levels of interferon-gamma, in a control individual,
wherein over expression or reduced expression or combination thereof of said gene(s) in combination with a reduced expression level of interferon-gamma as compared to corresponding gene expression levels and interferon gamma expression level in the control individual, provides a diagnosis of fibrotic interstitial lung disease in said individual.

34. The method of claim 33, wherein said pulmonary biological sample is lung biopsy, a lung aspirate or a combination thereof.

35. The method of claim 33, wherein said expression level of interferon-gamma is determined at the protein level or at the transcript level.

36. The method of claim 33, wherein said control individual is a healthy individual or an individual with a disease that is clinically similar to fibrotic interstitial lung disease.

37. The method of claim 33, wherein said genes that are overexpressed in fibrotic interstitial lung disease are selected from the group consisting of PTH-responsive osteosarcoma B1 protein, AF095771.1, matrix associated, actin dependent regulator of chromatin, subfamily f, member 1, AF231056.1, deleted in lung and esophageal cancer 1 (DLEC1), NM_{—}007337.1, major histocompatibility complex, class II, DQ beta 1, AW276186, SB classII histocompatibility antigen alpha-chain, AI128225, mucin 4, tracheobronchial, AJ242547.1, forkhead box J1 (FOXJ1), U69537.1, hypothetical protein FLJ21616, NM_{—}024567.1, neuronal specific transcription factor DAT1, AF258348.1, hematopoietic PBX-interacting protein, BF344265, proline oxidase homolog, AA074145, mucin 5, subtype B, tracheobronchial, AI697108, golgi membrane protein GP73, AF236056.1, ATP citrate lyase, U18197.1, NG22 protein, NM_{—}025257.1, cDNA DKFZp434A2322, AL137706.1, hepatocyte nuclear factor 3, alpha, U39840.1, major histocompatibility complex, class II, DQ alpha 1, X00452.1, myosin regulatory light chain 2, smooth muscle isoform, J02854.1, plexin B1, AV693216, pyruvate kinase, muscle, BC000481.1, tetraspanin TM4-C, AF133425.1, insulin-like growth factor binding protein 2 (36 kD), BC004312.1, FLJ13945 fis, clone Y79AA1000969, AU160041, hypothetical protein DKFZp586M1120, NM_{—}031294.1, CD24 signal transducer, L33930, hypothetical protein FLJ23571, NM_{—}025111.1, glutathione S-transferase M2 (muscle), M63509.1, cadherin 1, type 1, E-cadherin (epithelial), L08599.1, NTT5 protein, AF265578.1, lipocalin 2 (oncogene 24p3), NM_{—}005564.1, myotonic dystrophy kinase (DM kinase), L08835, uncoupling protein 2 (mitochondrial, proton carrier), U76367.1, dynein intermediate chain 2, NM_{—}023036.1, discoidin receptor tyrosine kinase isoform b, discoidin domain receptor family, member 1, NM_{—}001954.2, sperm associated antigen 6, AF079363.1, hypothetical protein FLJ23049, NM_{—}024687.1, nasopharyngeal epithelium specific protein 1, AF094758.1, nuclear receptor subfamily 4, group A, member 2, NM_{—}006186.1, hypothetical protein FLJ22215, BC003543.1, non-specific cross reacting antigen, M18728.1, amylase, alpha 1A; salivary (AMY1A), NM_{—}004038.1, carcinoembryonic antigen-related cell adhesion molecule 6 (non-specific cross reacting antigen), BC005008.1, glutathione S-transferase subunit 4 (EC 2.5.1.18), X08020.1, SH3-containing protein SH3GLB2, AF257319.1, KDEL (Lys-Asp-Glu-Leu) endoplasmic reticulum protein retention receptor 1, NM_{—}006801.1, anterior gradient 2 (Xenepus laevis) homolog, AF038451.1, sv7-MUC4 apomucin, mucin 4, tracheobronchial, AJ242547.1, stratifin, BC000329.1, connective tissue growth factor, M92934.1, cytochrome P450-IIB (hIIB3), M29873.1, filamin A, alpha (actin-binding protein-280), AW051856, membrane glycoprotein LIG-1, AB050468.1, E74-like factor 3 (ets domain transcription factor, epithelial-specific), U73844.1, elastin (supravalvular aortic stenosis, Williams-Beuren syndrome), M36860.1, ephrin receptor EPHA3, AF213459.1, fibrillin 1 (Marfan syndrome), L13923.1 discs, large (Drosophila) homolog 1, U13896.1, lysyl oxidase-like 1 (LOXL1), L21186.1, calumenin, U67280.1, male germ cell-associated kinase, NM_{—}005906.2, plectin 1, intermediate filament binding protein, 500 kD, Z54367, peroxisome biogenesis factor 1, AF026086.1, mast cell tryptase beta III, AF099143, ataxia-telangiectasia group D-associated protein, AF230388.1, hypothetical protein FLJ10921, NM 018272.1, biglycan, BC002416.1, BLu protein, AC002481, CGI-92 protein, AF151850.1, adaptor-related protein complex 1, mu 2 subunit, BC003387.1, keratin 15, BC002641.1, B7 protein, U72508.1, S100 calcium-binding protein A2, BC002829.1, MUF1 protein, 80004953.1, cholesterol 25-hydroxylase, AF059214.1, cytidine monophosphate-N-acetylneuraminic acid hydroxy-lase (CMP-N-acetylneuraminate monooxygenase), AF074480.1, neuropilin 2, AF022859.1, Fas-interacting serinethreonine kinase 3, homeodomain-interacting protein kinase 3, AF305239.1, a disintegrin and metalloproteinase domain 28 (ADAM28), transcript variant 2, AF137334.1, cDNA DKFZp434A119, AW663632, complement component 6, J05064.1, complement component 6, J05064.1, cytokeratin 17, Z19574, wingless-type MMTV integration site family, member 5A, AI968085, matrix metalloproteinase 7 (matrilysin, uterine), BC003635.1, leiomodin 1 (smooth muscle), NM_{—}012134.1, Cip1-interacting zinc finger protein, AB030835.1, cyclin-dependent kinase inhibitor 1A (p21, Cip1), BC000275.1, integrin, alpha 7, AF032108.1, DKFZP586G011 protein, BG289527, fatty acid binding protein 6, ileal (gastrotropin), U19869.1, glutathione S-transferase M4, M96234.1, Ras-related associated with diabetes, L24564.1, claudin 3, AB000714.1, matrix metalloproteinase 10 (stromelysin 2), BC002591.1, fibulin 2, NM_{—}001998.1, serine threonine kinase 11 (STK11), AF035625, eukaryotic translation initiation factor 1A: AF000987.1, DEADH (Asp-Glu-Ala-AspHis) box polypeptide: AF000985.1, ribosomal protein S4: AF116711.1, ubiquitin specific protease 9: AF000986.2, SMC (mouse) homolog: U52191.1, myelin basic protein: L18865.1, S100 calcium-binding protein: NM_{—}005980.1, Jagged2 (JAG2): AF003521.1, latent transforming growth factor beta binding protein 4: NM_{—}003573.1, microtubule-associated protein, RPEB family, member 3: AB025186.1, Unknown (protein for MGC:2854): BC003629.1, clone=IMAGE-2406340: AI830563, AQP3 gene for aquaporine 3 (water channel): AB001325, cDNA DKFZp434A119, fenestrated-endothelial linked structure protein (FELS), PV1 protein (PLVAP): AF326591.1, LUNX protein; PLUNC (palate lung and nasal epithelium clone); tracheal epithelium enriched protein (LOC51297): AB024937.1, RAB, member of RAS oncogene family-like 2A: AF095350.1, chromosome 11 open reading frame 16: NM_{—}020643.1, hypothetical protein FLJ23049: NM_{—}024687.1, hypothetical protein FLJ11767: NM_{—}024593.1, dynein, axonemal, intermediate polypeptide: AF091619.1, brain specific protein (LOC51673): AF132972.1, MUC4 apomucin, mucin 4, tracheobronchial: AJ242547.1, myotonin protein kinase (DM): M87313.1, cytokeratin 4: X07695.1, KIAA0362 gene, MCF.2 cell line derived transforming sequence-like: AB002360.1, cytokeratin 17: Z19574, LIM domain protein: BC003096.1, E74-like factor 3 (ets domain transcription factor, epithelial-specific): U73844.1, fatty acid binding protein 6, ileal (gastrotropin): U19869.1, sperm associated antigen 6: AF079363.1, eyes absent (Drosophila) homolog 2: U71207.1, phosphatidic acid phosphatase type 2C: BC002806.1, epoxide hydrolase 2, cytoplasmic: AF233334.1, tubulin, beta, 2: BC002783.1, heat shock 105 kD: D86956.1, heat shock 105 kD: D86956.1, villin 2 (ezrin): J05021.1, a disintegrin and metalloproteinase domain 28 (ADAM28), transcript variant 3: AF137335.1, deleted in lung and esophageal cancer 1: NM_{—}007337.1, arachidonate 15-lipoxygenase: NM_{—}001140.1, UDP glycosyltransferase 1 family, polypeptide A1: M57899.1, hypothetical protein PRO2834: AF119903.1, lectin, galactoside-binding, soluble, 7 (galectin 7): L07769.1, B7 protein: U72508.1, ephrin receptor EPHA3: AF213459.1, forkhead box J1: U69537.1, BLu protein: U70824.1, aldehyde dehydrogenase 3 family, member A1: BC004370.1, NG22 protein: NM_{—}025257.1, small inducible cytokine subfamily A (Cys-Cys), member 14: NM_{—}004166.1, cysteine-rich protein 1 (intestinal): BC002738.1, putative GTP-binding protein similar to RAYRABIC: BC000566.1, integrin, beta 4: NM_{—}000213.1, serine (or cysteine) proteinase inhibitor, clade B (ovalbumin), member 5 (SERPINB5): U04313.1, Ras-related associated with diabetes: L24564.1, hepatic leukemia factor: M95585.1, keratin 15: BC002641.1, nuclear receptor subfamily A, group A, member 2: NM_{—}006186.1, sialyltransferase: U14550.1, glutathione S-transferase M2 (muscle): M63509.1, hypothetical protein FLJ13110: NM_{—}022912.1, S100 calcium-binding protein A2: NM_{—}005978.2, collagen, type VII, alpha 1 (epidermolysis bullosa, dystrophic, dominant and recessive): L02870.1, claudin 3: AB000714.1, insulin-like growth factor binding protein 6: BC003507.1, fibroblast growth factor receptor 2 (bacteria-expressed kinase, keratinocyte growth factor receptor, craniofacial dysostosis 1, Crouzon syndrome, Pfeiffer syndrome, Jackson-Weiss syndrome): M80634.1, insulin-like growth factor 1 receptor: NM_{—}000875.2, insulin-like growth factor binding protein 2 (36 kD): M35410.1, ataxia-telangiectasia group D-associated protein: AF230388.1, keratin 5 (epidermolysis bullosa simplex, Dowling-MearaKobnerWeber-Cockayne types): M21389.1, Duffy blood group: U01839.1, transforming growth factor β1, NM_{—}000660

38. The method of claim 33, wherein said genes that are underexpressed in fibrotic interstitial lung disease are selected from a group consisting of sorting nexin 10 (SNX10), AF121860.1, phospholipase A2, group IIA (platelets, synovial fluid), M22430.1, hemoglobin alpha-1 globin chain (HBA1), AF349571.1, macrophage scavenger receptor 1, AI299239, surfactant, pulmonary-associated protein C, BC005913.1, disintegrin protease (M12.219), NM_{—}014479.1, retinol-binding protein 4, interstitial, AF119868.1, major histocompatibility complex, class II, DR beta 3, M27635.1, adipose differentiation-related protein, BC005127.1, hemoglobin, delta, NM_{—}000519.2, beta-2-microglobulin, AW188940, hemoglobin, alpha 2, BC005931.1, protein C receptor, endothelial (EPCR), L35545.1, thymosin, beta 10, M92381.1, apolipoprotein C-I, W79394, hypothetical protein, AL133067.1, proteoglycan 4, (megakaryocyte stimulating factor, articular superficial zone protein), U70136.1, tetranectin (plasminogen-binding protein), NM_{—}003278.1, platelet factor 4, M25897.1, tissue factor pathway inhibitor 2, BC005330.1, fatty acid binding protein 4, adipocyte, BC003672.1, cathepsin B (CTSB), M14221.1, transmembrane 4 superfamily member 1, M90657.1, MHC class I HLA-B51 major histocompatibility complex, class I, E, M31183.1, endothelial PAS domain protein 1, U51626.1, Apo-2 ligand tumor necrosis factor (ligand) superfamily, member 10, U37518.1, haptoglobin-related protein, NM_{—}020995.1, Rho guanine exchange factor (GEF) 12, AF119898.1, major histocompatibility complex, class II, DR beta 5, M11867.1, interferon, gamma-inducible protein 30, AF097362.1, ferritin, light polypeptide, BG537190, prosaposin (variant Gaucher disease and variant metachromatic leukodystrophy), BC004275.1, calcium-binding protein A4 (calvasculin, metastasin,), NM_{—}002961.2, hemoglobin, beta, M25079.1, CDW52 antigen (CAMPATH-1 antigen), BC000644.1, aldehyde dehydrogenase 1 family, member A2, AB015226.1, cathepsin Z, AF032906.1, MHC HLA-B39 major histocompatibility complex, class I, B, L37880.1, major histocompatibility complex, class II, DQ alpha 1, M33906.1, fibroblast growth factor 9 (glia-activating factor), D14838.1, hemoglobin, alpha 2, AF097635.1, transferrin receptor (p90, CD71), BC001188.1, complement component 3 (C3), K02765.1, cDNA DKFZp564D066, AL050025.1, complement component 1, q subcomponent, beta polypeptide NM_{—}000491.2, small inducible cytokine subfamily A (Cys-Cys), member 18, pulmonary and activation-regulated, AB000221.1, reticulon 1, L10333.1, major histocompatibility complex, class II, DR beta 1, M33600.1, haptoglobin, L29394.1, acid phosphatase 5, tartrate resistant, J04430.1, cytochrome P450, subfamily XXVIIA (steroid 27-hydroxylase, cerebrotendinous xanthomatosis), polypeptide 1, M62401.1, CD36 antigen (collagen type I receptor, thrombospondin receptor), M24795.1, calbindin 2, (29 kD, calretinin), NM_{—}001740.2, alpha-2-HS-glycoprotein, BG538564, cDNA DKFZp564A132, AL049963.1, fibronectin 1, AF130095.1, phosphodiesterase 4C, cAMP-specific, NM_{—}000923.1, transcription factor 7 (T-cell specific, HMG-box), N_{—}003202.1, found in inflammatory zone 3 (FIZZ3), AF323081.1, claudin 15, NM_{—}014343.1, carboxypeptidase B1 (tissue), M81057.1, hypothetical protein FLJ14054, NM_{—}024563.1, bone marrow stromal cell antigen 1, D21878.1, interleukin 7 receptor, M29696.1, procollagen C-endopeptidase enhancer 2, AF098269.1, calcium-binding protein A8 (calgranulin A), AW238654, cDNA DKFZp564D193, AL049252.1, major histocompatibility complex, class II, P beta 1, J03041.1, human leukocyte antigen C alpha chain, major histocompatibility complex, class I, C, AK024836.1, BCM-like membrane protein precursor, AF144235.1, CD14 antigen, M86511.1, pulmonary surfactant protein (SP5), J03553, signal transducer and activator of transcription 1, 91 kD, BC002704.1, Wilms tumor 1 (WT1), transcript variant D, NM_{—}024424.1, annexin A8, BC004376.1, macrophage receptor with collagenous structure, MARCO , AF035819.1, surfactant, pulmonary-associated protein A2, NM_{—}006926.1, solute carrier family 6 (neurotransmitter transporter, serotonin), member 4, L05568.1, chitinase 1 (chitotriosidase), U29615.1, lung type-I cell membrane-associated glycoprotein, AU154455, fibronectin leucine rich transmembrane protein 2, AB007865.1, gamma-aminobutyric acid (GABA) A receptor, alpha 5, B966183, hypothetical protein FLJ12983, NM_{—}024856.1, sialophorin (gpL115, leukosialin, CD43), J04536.1, cerebellar degeneration-related protein (34 kD), M16965.1, hydroxyacid oxidase 2 (long chain), hydroxy-delta-5-steroid dehydrogenase (3 beta- and steroid delta-isomeras 2), AL359553, CLONE=IMAGE:1032795=Hs.83623 nuclear receptor subfamily 1, group I, member 3, small inducible cytokine subfamily C, member 2: NM_{—}003175.1, hypothetical protein similar to swine acylneuraminate lyase: NM_{—}030769.1, fibrinogen, gamma polypeptide: AF118092.1, thrombospondin 1: NM_{—}003246.1, SAM domain, SH3 domain and nuclear localisation signals, 1: AF222927.1, chitinase 3-like 1 (cartilage glycoprotein-39): M80927.1, cathepsin Z: AF032906.1, CLONE=IMAGE:3579023 collagen, type XIV, alpha 1 (undulin), chloride intracellular channel 2: NM_{—}001289.2, monokine induced by gamma interferon: NM_{—}002416.1, KIAA0433 protein: NM_{—}015216.1, tumor necrosis factor, alpha-induced protein 6: NM_{—}007115.1, signal transducer and activator of transcription 1, 91 kD: BC002704.1, thrombospondin 2: L12350.1, collagen, type IV, alpha 3 (Goodpasture antigen): NM_{—}000091.1, integrin, beta-like 1 (with EGF-like repeat domains): AF072752.1, diubiquitin: NM_{—}006398.1, protease, cysteine, 1 (legumain): D55696.1, cytochrome P450, subfamily I (dioxin-inducible), polypeptide 1 (glaucoma 3, primary infantile): U03688.1, integrin, alpha 1: X68742.1, GABA-B receptor, G protein-coupled receptor 51: AF056085.1, KIAA1199 protein: AB033025.1, collagen, type V, alpha 2: NM_{—}000393.1, interleukin 13 receptor, alpha 2: U70981.1, translocase of inner mitochondrial membrane 8 (yeast) homolog A: BC005236.1, steroid sulfatase (microsomal), arylsulfatase C, isozyme S: M16505.1, CLONE=IMAGE:1982571 ATPase, H+ transporting, lysosomal (vacuolar proton pump) 9 kD, proteoglycan 4, (megakaryocyte stimulating factor, articular superficial zone protein): U70136.1, nidogen (enactin): M30269.1, KIAA1598 protein: AU157109, vascular cell adhesion molecule 1: M60335.1, guanylate binding protein 1, interferon-inducible, 67 kD: BC002666.1, cDNA DKFZp586E1124, apolipoprotein H (beta-2-glycoprotein I): M62839.1, ribosomal protein L37a: BE857772, cDNA DKFZp564A132, cathepsin S: M86553.1, a disintegrin and metalloproteinase domain 9 (meltrin gamma) (ADAM9): U41766.1, zinc finger protein 331: AF272148.1, lysosomal-associated membrane protein 2: J04183.1, carboxypeptidase M: NM_{—}001874.1, collagen, type I, alpha 2: N_{—}000089.1, P311 protein: U36189.1, KIAA0372 gene product: AB002370.1, Human T cell-specific protein RANTES: M21121, interferon-gamma-inducible indoleamine 2,3-dioxygenase (IDO): M34455.1, hypothetical protein FLJ10430: NM_{—}018092.1, transcription factor ISGF-3: M97935, interleukin 1 receptor-like 1 (IL1RL1): NM_{—}003856.1, putative alpha chemokine (H174), small inducible cytokine subfamily B (Cys-X-Cys), member II: AF002985.1, small inducible cytokine subfamily B (Cys-X-Cys), member 10: NM_{—}001565.1, mesoderm specific transcript (mouse) homolog: BC002413.1, carboxypeptidase B-like protein: AB011969.1, CD2 antigen p50), sheep red blood cell receptor: M16445, interferon, alpha-inducible protein (clone IFI-6-16): NM_{—}022872.1, RAS guanyl releasing protein 1 (calcium and DAG-regulated): AF081195.1, lipase, endothelial: AF118767.1, fatty-acid-Coenzyme A ligase, long-chain 4: NM_{—}022977.1, klotho: AB005142.1, chondroitin sulfate proteoglycan 2 (versican): NM_{—}004385.1, hypothetical protein, expressed in osteoblast: AB 000115.1, CD14 antigen: M86511.1, CGI-83 protein: BC000878.1, leucine aminopeptidase: AF061738.1, UDP-Gal:betaGlcNAc beta 1,3-galactosyltransferase, polypeptide 3: AB050855.1, protocadherin alpha 12: AF152308.1, neuroglycan C: AF059274, neuroligin: AI338338, HSPC156 protein: AF161505.1, hypothetical protein FLJ13310: NM_{—}025118.1, eosinophil chemotactic cytokine (TSA1902): AB025008.1 aminopeptidase: AF191545.1, semaphorin sem2: AB029496.1, protocadherin 12: AF231025.1, X transporter protein 3: NM_{—}020208.1, transmembrane 4 superfamily member (tetraspan NET-2): AF124522.1, hypothetical protein FLJ10970: NM_{—}018286.1, perforin 1 (pore forming protein): M28393.1, natural killer cell group 7 sequence: NM_{—}005601.1, hypothetical protein DKFZp761N09121: BF435376, integrin, alpha 4 (antigen CD49D, alpha 4 subunit of VLA-4 receptor): BG532690, chitinase 3-like 2: U58515.1, FYN oncogene: N20923, relaxin 1 (H1): BC005956.1, hydroxyprostaglandin dehydrogenase 15-(NAD): U63296.1, sulfotransferase family, cytosolic, 1C, member 1: AF186254.1, TGF-b superfamily receptor type I: L17075.1, granzyme B (granzyme 2, cytotoxic T-lymphocyte-associated serine esterase 1): M36118.1, cystatin F (leukocystatin): AF031824.1, regulator of G protein signaling-Z (RGSZ1): AF060877.2, clone MGC:12387: M16942.1, zinc-alpha2-glycoprotein: D90427.1, BCG induced integral membrane protein BIGMo-103: AB040120.1, nectin-like protein 2 (NECL2): AF132811.1, CD209 antigen-like: AB015629.1, solute carrier family 6 (neurotransmitter transporter, serotonin), member 4: L05568.1, MAD (mothers against decapentaplegic, Drosophila) homolog 6: U59914.1, latrophilin: AF104939.1, platelet factor 4: M25897.1, calcitonin receptor-like: L76380.1, matrilin 3: NM_{—}002381.2, solute carrier family 1 (urea transporter), member 1 (Kidd blood group): U35735.1, interleukin 7 receptor: M29696.1, MAP kinase kinase 6 (MKK6), mitogen-activated protein kinase kinase 6: U39656.1, protocadherin 17: AF029343.1, granulysin: NM_{—}006433.2, interferon-stimulated protein, 15 kDa (ISG15): M13755.1, cadherin 5, type 2, VE-cadherin (vascularepithelium): U84722.1, thrombomodulin: M16552.1, interferon, alpha-inducible protein 27: NM_{—}005532.1, interferon-gamma, X87308

39. The method of claim 33, wherein said gene expression is determined by DNA microarray, Protein microarray, or RT-PCR.

40. The method of claim 33, wherein said fibrotic interstitial lung disease is idiopathic pulmonary fibrosis (IPF), hypersensivity pneumonitis, scleroderma, Systemic Lupus Erythematosus, Rheumatoid Arthritis, Churg-Strauss syndrome, Wegener's granulomatosis, or Goodpasture Syndrome.

41. A method of treatment for Fibrotic Interstitial Lung Disease in an individual in need thereof comprising:
obtaining a pulmonary biological sample from said individual;
determining expression levels of genes that are either over expressed or under expressed in fibrotic interstitial lung disease, in comparison to corresponding gene expression levels in a control individual, in said sample;
determining interferon-gamma deficiency in said sample;
detecting underlying infection in said sample;
treating said underlying infection in the individual; and
administering interferon-gamma or pirfenidone.

42. The method of claim 41, further comprising administration of glucocorticoids.

43. The method of claim 42, wherein said glucocorticoid is administered in a dose ranging from 100 to 350 microgram/kg body weight once a week.

44. The method of claim 41, wherein said pulmonary biological sample is a lung biopsy, a lung aspirate or a combination thereof.

45. The method of claim 41, wherein said genes that are overexpressed are selected from the group consisting of PTH-responsive osteosarcoma B1 protein, AF095771.1, matrix associated, actin dependent regulator of chromatin, subfamily f, member 1, AF231056.1, deleted in lung and esophageal cancer 1 (DLEC1), NM_{—}007337.1, major histocompatibility complex, class II, DQ beta 1, AW276186, SB classII histocompatibility antigen alpha-chain, AI128225, mucin 4, tracheobronchial, AJ242547.1, forkhead box J1 (FOXJ1), U69537.1, hypothetical protein FLJ21616, NM_{—}024567.1, neuronal specific transcription factor DAT1, AF258348.1, hematopoietic PBX-interacting protein, BF344265, proline oxidase homolog, AA074145, mucin 5, subtype B, tracheobronchial, A697108, golgi membrane protein GP73, AF236056.1, ATP citrate lyase, U18197.1, G22 protein, NM_{—}025257.1, cDNA DKFZp434A2322, AL137706.1, hepatocyte nuclear factor 3, alpha, U39840.1, major histocompatibility complex, class II, DQ alpha 1, X00452.1, myosin regulatory light chain 2, smooth muscle isoform, J02854.1, plexin B1, AV693216, pyruvate kinase, muscle, BC000481.1, tetraspanin TM4-C, AF133425.1, insulin-like growth factor binding protein 2 (36 kD), BC004312.1, FLJ13945 fis, clone Y79AA1000969, AU160041, hypothetical protein DKFZp586M1120, NM_{—}031294.1, CD24 signal transducer, L33930, hypothetical protein FLJ23571, NM_{—}025111.1, glutathione S-transferase M2 (muscle), M63509.1, cadherin 1, type 1, E-cadherin (epithelial), L08599.1, NTT5 protein, AF265578.1, lipocalin 2 (oncogene 24p3), NM_{—}005564.1, myotonic dystrophy kinase (DM kinase), L08835, uncoupling protein 2 (mitochondrial, proton carrier), U76367.1, dynein intermediate chain 2, NM_{—}023036.1, discoidin receptor tyrosine kinase isoform b, discoidin domain receptor family, member 1, NM_{—}001954.2, sperm associated antigen 6, AF079363.1, hypothetical protein FLJ23049, NM_{—}024687.1, nasopharyngeal epithelium specific protein 1, AF094758.1, nuclear receptor subfamily 4, group A, member 2, NM_{—}006186.1, hypothetical protein FLJ22215, BC003543.1, non-specific cross reacting antigen, M18728.1, amylase, alpha 1A; salivary (AMY1A), NM_{—}004038.1, carcinoembryonic antigen-related cell adhesion molecule 6 (non-specific cross reacting antigen), BC005008.1, glutathione S-transferase subunit 4 (EC 2.5.1.18), X08020.1, SH3-containing protein SH3GLB2, AF257319.1, KDEL (Lys-Asp-Glu-Leu) endoplasmic reticulum protein retention receptor 1, NM_{—}006801.1, anterior gradient 2 (Xenepus laevis) homolog, AF038451.1, sv7-MUC4 apomucin, mucin 4, tracheobronchial, AJ242547.1, stratifin, BC000329.1, connective tissue growth factor, M92934.1, cytochrome P450-IIB (hIIB3), M29873.1, filamin A, alpha actin-binding protein-280), AW051856, membrane glycoprotein LIG-1, AB050468.1, E74-like factor 3 (ets domain transcription factor, epithelial-specific), U73844.1, elastin (supravalvular aortic stenosis, Williams-Beuren syndrome), M36860.1, ephrin receptor EPHA3, AF213459.1, fibrillin 1 Marfan syndrome), L13923.1 discs, large (Drosophila) homolog 1, U13896.1, lysyl oxidase-like 1 (LOXL1), L21186.1, calumenin, U67280.1, male germ cell-associated kinase, NM_{—}005906.2, plectin 1, intermediate filament binding protein, 500 kD, Z54367, peroxisome biogenesis factor 1, AF026086.1, mast cell tryptase beta III, AF099143, ataxia-telangiectasia group D-associated protein, AF230388.1, hypothetical protein FLJ10921, NM_{—}018272.1, biglycan, BC002416.1, BLu protein, AC002481, CGI-92 protein, AF151850.1, adaptor-related protein complex 1, mu 2 subunit, BC003387.1, keratin 15, BC002641.1, B7 protein, U72508.1, S100 calcium-binding protein A2, BC002829.1, MUF1 protein, 80004953.1, cholesterol 25-hydroxylase, AF059214.1, cytidine monophosphate-N-acetylneuraminic acid hydroxy-lase (CMP-N-acetylneuraminate monooxygenase), AF074480.1, neuropilin 2, AF022859.1, Fas-interacting serinethreonine kinase 3, homeodomain-interacting protein kinase 3, AF305239.1, a disintegrin and metalloproteinase domain 28 (ADAM28), transcript variant 2, AF137334.1, cDNA DKFZp434 A119, AW663632, complement component 6, J05064.1, complement component 6, J05064.1, cytokeratin 17, Z19574, wingless-type MMTV integration site family, member 5A, AI968085, matrix metalloproteinase 7 (matrilysin, uterine), BC003635.1, leiomodin 1 (smooth muscle), NM_{—}012134.1, Cip1-interacting zinc finger protein, AB030835.1, cyclin-dependent kinase inhibitor 1A (p21, Cip1), BC000275.1, integrin, alpha 7, AF032108.1, DKFZP586G011 protein, BG289527, fatty acid binding protein 6, ileal (gastrotropin), U19869.1, glutathione S-transferase M4, M96234.1, Ras-related associated with diabetes, L24564.1, claudin 3, AB000714.1, matrix metalloproteinase 10 (stromelysin 2), BC002591.1, fibulin 2, NM 001998.1, serine threonine kinase 11 (STK11), AF035625, eukaryotic translation initiation factor 1A: AF000987.1, DEADH (Asp-Glu-Ala-AspHis) box polypeptide: AF000985.1, ribosomal protein S4: AF116711.1, ubiquitin specific protease 9: AF000986.2, SMC (mouse) homolog: U52191.1, myelin basic protein: L18865.1, S100 calcium-binding protein: NM_{—}005980.1, Jagged2 (JAG2): AF003521.1, latent transforming growth factor beta binding protein 4: NM_{—}003573.1, microtubule-associated protein, RPEB family, member 3: AB025186.1, Unknown (protein for MGC:2854): BC003629.1, clone=IMAGE-2406340: AI830563, AQP3 gene for aquaporine 3 (water channel): AB001325, cDNA DKFZp434A119, fenestrated-endothelial linked structure protein (FELS), PV1 protein (PLVAP): AF326591.1, LUNX protein; PLUNC (palate lung and nasal epithelium clone); tracheal epithelium enriched protein (LOC51297): AB024937.1, RAB, member of RAS oncogene family-like 2A: AF095350.1, chromosome 11 open reading frame 16: NM_{—}020643.1, hypothetical protein FLJ23049: NM_{—}024687.1, hypothetical protein FLJ11767: NM_{—}024593.1, dynein, axonemal, intermediate polypeptide: AF091619.1, brain specific protein (LOC51673): AF132972.1, MUC4 apomucin, mucin 4, tracheobronchial: AJ242547.1, myotonin protein kinase (DM): M87313.1, cytokeratin 4: X07695.1, KIAA0362 gene, MCF.2 cell line derived transforming sequence-like: AB002360.1, cytokeratin 17: Z19574, LIM domain protein: BC003096.1, E74-like factor 3 (ets domain transcription factor, epithelial-specific): U73844.1, fatty acid binding protein 6, ileal (gastrotropin): U19869.1, sperm associated antigen 6: AF079363.1, eyes absent (Drosophila) homolog 2: U71207.1, phosphatidic acid phosphatase type 2C: BC002806.1, epoxide hydrolase 2, cytoplasmic: AF233334.1, tubulin, beta, 2: BC002783.1, heat shock 105 kD: D86956.1, heat shock 105 kD: D86956.1, villin 2 (ezrin): J05021.1, a disintegrin and metalloproteinase domain 28 (ADAM28), transcript variant 3: AF137335.1, deleted in lung and esophageal cancer 1: NM_{—}007337.1, arachidonate 15-lipoxygenase: NM_{—}001140.1, UDP glycosyltransferase 1 family, polypeptide A1: M57899.1, hypothetical protein PRO2834: AF119903.1, lectin, galactoside-binding, soluble, 7 (galectin 7): L07769.1, B7 protein: U72508.1, ephrin receptor EPHA3: AF213459.1, forkhead box J1: U69537.1, BLu protein: U70824.1, aldehyde dehydrogenase 3 family, member A1: BC004370.1, NG22 protein: NM_{—}025257.1, small inducible cytokine subfamily A (Cys-Cys), member 14: NM_{—}004166.1, cysteine-rich protein 1 (intestinal): BC002738.1, putative GTP-binding protein similar to RAYRAB1C: BC000566.1, integrin, beta 4: NM_{—}000213.1, serine (or cysteine) proteinase inhibitor, clade B (ovalbumin), member 5 (SERPINB5): U04313.1, Ras-related associated with diabetes: L24564.1, hepatic leukemia factor: M95585.1, keratin 15: BC002641.1, nuclear receptor subfamily 4, group A, member 2: NM_{—}006186.1, sialyltransferase: U14550.1, glutathione S-transferase M2 (muscle): M63509.1, hypothetical protein FLJ13110: NM_{—}022912.1, S100 calcium-binding protein A2: NM_{—}005978.2, collagen, type VII, alpha 1 (epidermolysis bullosa, dystrophic, dominant and recessive): L02870.1, claudin 3: AB000714.1, insulin-like growth factor binding protein 6: BC003507.1, fibroblast growth factor receptor 2 (bacteria-expressed kinase, keratinocyte growth factor receptor, craniofacial dysostosis 1, Crouzon syndrome, Pfeiffer syndrome, Jackson-Weiss syndrome): M80634.1, insulin-like growth factor 1 receptor: NM_{—}000875.2, insulin-like growth factor binding protein 2 (36 kD): M35410.1, ataxia-telangiectasia group D-associated protein: AF230388.1, keratin 5 (epidermolysis bullosa simplex, Dowling-MearaKobnerWeber-Cockayne types): M21389.1, Duffy blood group: U01839.1, transforming growth factor β₁, NM_{—}000660

46. The method of claim 41, wherein said genes that are under expressed are selected from the group consisting of sorting nexin 10 (SNX10), AF 121860.1, phospholipase A2, group IIA (platelets, synovial fluid), M22430.1, hemoglobin alpha-1 globin chain (HBA1), AF349571.1, macrophage scavenger receptor 1, AI299239, surfactant, pulmonary-associated protein C, BC005913.1, disintegrin protease (M12.219), NM_{—}014479.1, retinol-binding protein 4, interstitial, AF119868.1, major histocompatibility complex, class II, DR beta 3, M27635.1, adipose differentiation-related protein, BC005127.1, hemoglobin, delta, NM_{—}000519.2, beta-2-microglobulin, AW188940, hemoglobin, alpha 2, BC005931.1, protein C receptor, endothelial (EPCR), L35545.1, thymosin, beta 10, M92381.1, apolipoprotein C-I, W79394, hypothetical protein, AL133067.1, proteoglycan 4, (megakaryocyte stimulating factor, articular superficial zone protein), U70136.1, tetranectin (plasminogen-binding protein), NM_{—}003278.1, platelet factor 4, M25897.1, tissue factor pathway inhibitor 2, BC005330.1, fatty acid binding protein 4, adipocyte, BC003672.1, cathepsin B (CTSB), M14221.1, transmembrane 4 superfamily member 1, M90657.1, MHC class I HLA-B51 major histocompatibility complex, class I, E, M31183.1, endothelial PAS domain protein 1, U51626.1, Apo-2 ligand tumor necrosis factor (ligand) superfamily, member 10, U37518.1, haptoglobin-related protein, NM_{—}020995.1, Rho guanine exchange factor (GEF) 12, AF119898.1, major histocompatibility complex, class II, DR beta 5, M11867.1, interferon, gamma-inducible protein 30, AF097362.1, ferritin, light polypeptide, BG537190, prosaposin (variant Gaucher disease and variant metachromatic leukodystrophy), BC004275.1, calcium-binding protein A4 (calvasculin, metastasin,), NM_{—}002961.2, hemoglobin, beta, M25079.1, CDW52 antigen (CAMPATH-1 antigen), BC000644.1, aldehyde dehydrogenase 1 family, member A2, AB015226.1, cathepsin Z, AF032906.1, MHC HLA-B39 major histocompatibility complex, class I, B, L37880.1, major histocompatibility complex, class II, DQ alpha 1, M33906.1, fibroblast growth factor 9 (glia-activating factor), D14838.1, hemoglobin, alpha 2, AF097635.1, transferrin receptor (p90, CD71), BC001188.1, complement component 3 (C3), K02765.1, cDNA DKFZp564D066, AL050025.1, complement component 1, q subcomponent, beta polypeptide NM_{—}000491.2, small inducible cytokine subfamily A (Cys-Cys), member 18, pulmonary and activation-regulated, AB000221.1, reticulon 1, L10333.1, major histocompatibility complex, class II, DR beta 1, M33600.1, haptoglobin, L29394.1, acid phosphatase 5, tartrate resistant, J04430.1, cytochrome P450, subfamily XXVIIA (steroid 27-hydroxylase, cerebrotendinous xanthomatosis), polypeptide 1, M62401.1, CD36 antigen (collagen type I receptor, thrombospondin receptor), M24795.1, calbindin 2, (29 kD, calretinin), NM_{—}001740.2, alpha-2-HS-glycoprotein, BG538564, cDNA DKFZp564A132, AL049963.1, fibronectin 1, AF130095.1, phosphodiesterase 4C, cAMP-specific, NM_{—}000923.1, transcription factor 7 (T-cell specific, HMG-box), NM_{—}003202.1, found in inflammatory zone 3 (FIZZ3), AF323081.1, claudin 15, NM_{—}014343.1, carboxypeptidase B1 (tissue), M81057.1, hypothetical protein FLJ14054, NM_{—}024563.1, bone marrow stromal cell antigen 1, D21878.1, interleukin 7 receptor, M29696.1, procollagen C-endopeptidase enhancer 2, AF098269.1, calcium-binding protein A8 (calgranulin A), AW238654, cDNA DKFZp564D193, AL049252.1, major histocompatibility complex, class II, DP beta 1, J03041.1, human leukocyte antigen C alpha chain, major histocompatibility complex, class I, C, AK024836.1, BCM-like membrane protein precursor, AF144235.1, CD14 antigen, M86511.1, pulmonary surfactant protein (SP5), J03553, signal transducer and activator of transcription 1, 91 kD, BC002704.1, Wilms tumor 1 (WT1), transcript variant D, NM_{—}024424.1, annexin A8, BC004376.1, macrophage receptor with collagenous structure, MARC0, AF035819.1, surfactant, pulmonary-associated protein A2, NM_{—}006926.1, solute carrier family 6 (neurotransmitter transporter, serotonin), member 4, L05568.1, chitinase 1 (chitotriosidase), U29615.1, lung type-I cell membrane-associated glycoprotein, AU154455, fibronectin leucine rich transmembrane protein 2, AB007865.1, gamma-aminobutyric acid (GABA) A receptor, alpha 5, BF966183, hypothetical protein FLJ12983, NM_{—}024856.1, sialophorin (gpL115, leukosialin, CD43), J04536.1, cerebellar degeneration-related protein (34 kD), M16965.1, hydroxyacid oxidase 2 (long chain), hydroxy-delta-5-steroid dehydrogenase (3 beta- and steroid delta-isomerase 2), AL359553, CLONE=IMAGE:1032795=Hs.83623 nuclear receptor subfamily 1, group I, member 3, small inducible cytokine subfamily C, member 2: NM_{—}003175.1, hypothetical protein similar to swine acylneuraminate lyase: NM_{—}030769.1, fibrinogen, gamma polypeptide: AF118092.1, thrombospondin 1: NM_{—}003246.1, SAM domain, SH3 domain and nuclear localisation signals, 1: AF222927.1, chitinase 3-like 1 (cartilage glycoprotein-39): M80927.1, cathepsin Z: AF032906.1, CLONE=IMAGE:3579023 collagen, type XIV, alpha 1 (undulin), chloride intracellular channel 2: NM_{—}001289.2, monokine induced by gamma interferon: NM_{—}002416.1, KIAA0433 protein: NM_{—}015216.1, tumor necrosis factor, alpha-induced protein 6: NM_{—}007115.1, signal transducer and activator of transcription 1, 91 kD: BC002704.1, thrombospondin 2: L12350.1, collagen, type IV, alpha 3 (Goodpasture antigen): NM_{—}000091.1, integrin, beta-like 1 (with EGF-like repeat domains): AF072752.1, diubiquitin: NM_{—}006398.1, protease, cysteine, 1 (legumain): D55696.1, cytochrome P450, subfamily I (dioxin-inducible), polypeptide 1 (glaucoma 3, primary infantile): U03688.1, integrin, alpha 1: X68742.1, GABA-B receptor, G protein-coupled receptor 51: AF056085.1, KIAA1199 protein: AB033025.1, collagen, type V, alpha 2: NM_{—}000393.1, interleukin 13 receptor, alpha 2: U70981.1, translocase of inner mitochondrial membrane 8 (yeast) homolog A: BC005236.1, steroid sulfatase (microsomal), arylsulfatase C, isozyme S: M16505.1, CLONE=IMAGE:1982571 ATPase, H+ transporting, lysosomal (vacuolar proton pump) 9 kD, proteoglycan 4, (megakaryocyte stimulating factor, articular superficial zone protein): U70136.1, nidogen (enactin): M30269.1, KIAA1598 protein: AU157109, vascular cell adhesion molecule 1: M60335.1, guanylate binding protein 1, interferon-inducible, 67 kD: BC002666.1, cDNA DKFZp586E1124, apolipoprotein H (beta-2-glycoprotein I): M62839.1, ribosomal protein L37a: BE857772, cDNA DKFZp564A132, cathepsin S: M86553.1, a disintegrin and metalloproteinase domain 9 (meltrin gamma) (ADAM9): U41766.1, zinc finger protein 331: AF272148.1, lysosomal-associated membrane protein 2: J04183.1, carboxypeptidase M: NM_{—}001874.1, collagen, type I, alpha 2: NM_{—}000089.1, P311 protein: U36189.1, KIAA0372 gene product: AB002370.1, Human T cell-specific protein RANTES: M21121, interferon-gamma-inducible indoleamine 2,3-dioxygenase (IDO): M34455.1, hypothetical protein FLJ10430: NM_{—}018092.1, transcription factor ISGF-3: M97935, interleukin 1 receptor-like 1 (IL1RL1): NM_{—}003856.1, putative alpha chemokine (H174), small inducible cytokine subfamily B (Cys-X-Cys), member 11: AF002985.1, small inducible cytokine subfamily B (Cys-X-Cys), member 10: NM_{—}001565.1, mesoderm specific transcript (mouse) homolog: BC002413.1, carboxypeptidase B-like protein: AB011969.1, CD2 antigen (p50), sheep red blood cell receptor: M16445, interferon, alpha-inducible protein (clone IFI-6-16): NM_{—}022872.1, RAS guanyl releasing protein 1 (calcium and DAG-regulated): AF081195.1, lipase, endothelial: AF118767.1, fatty-acid-Coenzyme A ligase, long-chain 4: NM_{—}022977.1, klotho: AB005142.1, chondroitin sulfate proteoglycan 2 (versican): NM_{—}004385.1, hypothetical protein, expressed in osteoblast: AB00115.1, CD14 antigen: M86511.1, CGI-83 protein: BC000878.1, leucine aminopeptidase: AF061738.1, UDP-Gal:betaGlcNAc beta 1,3-galactosyltransferase, polypeptide 3: AB050855.1, protocadherin alpha 12: AF152308.1, neuroglycan C: AF059274, neuroligin: AI338338, HSPC156 protein: AF161505.1, hypothetical protein FLJ13310: NM_{—}025118.1, eosinophil chemotactic cytokine (TSA1902): AB025008.1, aminopeptidase: AF191545.1, semaphorin sem2: AB029496.1, protocadherin 12: AF231025.1, X transporter protein 3: NM_{—}020208.1, transmembrane 4 superfamily member (tetraspan NET-2): AF124522.1, hypothetical protein FLJ10970: NM_{—}018286.1, perforin 1 (pore forming protein): M28393.1, natural killer cell group 7 sequence: NM_{—}005601.1, hypothetical protein DKFZp761N09121: BF435376, integrin, alpha 4 (antigen CD49D, alpha 4 subunit of VLA-4 receptor): BG532690, chitinase 3-like 2: U58515.1, FYN oncogene: N20923, relaxin 1 (H1): BC005956.1, hydroxyprostaglandin dehydrogenase 15-(NAD): U63296.1, sulfotransferase family, cytosolic, 1C, member 1: AF186254.1, TGF-b superfamily receptor type I: L17075.1, granzyme B (granzyme 2, cytotoxic T-lymphocyte-associated serine esterase 1): M36118.1, cystatin F (leukocystatin): AF031824.1, regulator of G protein signaling-Z (RGSZ1): AF060877.2, clone MGC:12387: M16942.1, zinc-alpha2-glycoprotein: D90427.1, BCG induced integral membrane protein BIGMo-103: AB040120.1, nectin-like protein 2 (NECL2): AF132811.1, CD209 antigen-like: AB015629.1, solute carrier family 6 (neurotransmitter transporter, serotonin), member 4: L05568.1, MAD (mothers against decapentaplegic, Drosophila) homolog 6: U59914.1, latrophilin: AF104939.1, platelet factor 4: M25897.1, calcitonin receptor-like: L76380.1, matrilin 3: NM_{—}002381.2, solute carrier family 14 (urea transporter), member 1 (Kidd blood group): U35735.1, interleukin 7 receptor: M29696.1, MAP kinase kinase 6 (MKK6), mitogen-activated protein kinase kinase 6: U39656.1, protocadherin 17: AF029343.1, granulysin: NM_{—}006433.2, interferon-stimulated protein, 15 kDa (ISG15): M13755.1, cadherin 5, type 2, VE-cadherin (vascularepithelium): U84722.1, thrombomodulin: M16552.1, interferon, alpha-inducible protein 27: NM_{—}005532.1, interferon-gamma, X87308

47. The method of claim 41, wherein said interferon-gamma deficiency is characterized by decreased expression levels of interferon-gamma as compared to expression levels of interferon-gamma in a control individual.

48. The method of claim 47, wherein said interferon-gamma expression is detected at the protein or transcript level.

49. The method of claims 41 or 47, wherein said control individual is a healthy individual or an individual with disease clinically similar to fibrotic interstitial lung disease.

50. The method of claim 41, wherein said underlying infection is detected by identifying gene expression or genomic DNA of the infective agent in the biological sample.

51. The method of claim 50, wherein said infective agent is a bacteria, yeast, fungus or a virus.

52. The method of claim 51, wherein said bacteria is selected from the group consisting of Mycobacterium, Mycoplasma, Staphyllococcus aureus, Streptococcus, Borrelia, Treponema pallidum, Leptospira interrogans, Campylobacter jejuni, Campylobacter fetus; Escherichia coli, EPEC, ETEC, EIEC, EHEC Salmonella enterica, Yersinia enterocolitica, Aeromonas, Moraxella catarrhalis, Brucella, Toxoplasma, Salmonella enterica, Shigella spec., Yersinia enterocolitica, Vibrio cholerae, Pseudomonas aeruginosa, Burkholderia cepacia, Stenotrophomonas maltophilia, Acinetobacter baumanii, Acitenobacter calcoaceticus, Klebsiella, Enterobacter, Citrobacter, Proteus, Serratia, Morganella, Providencia, Cardiobacterium hominis, Eikenella corrodens, Gardnerella vaginalis, Calymmatobacterium granulomatis, Bacteriodes, Porphyromonas, Prevotella, Fusobacterium, Rickettsia prowazekii, Bartonella bacilliformis, Bartonella henselae or Chlamydia.

53. The method of claim 51, wherein said virus is selected from the group consisting of retroviruses, adenoviruses, hepadnaviruses, herpesviruses, influenza viruses, or paramyxoviruses.

54. The method of claim 41, wherein said treatment of underlying infection is by administration of antibiotics, anti-fungals and antivirals.

55. The method of claim 41, wherein said interferron-gamma is administered in a dose ranging from 1.0 to 5.0 micrograms/kg body weight one to five times a week.

56. The method of claim 41, wherein said interferron-gamma is administered in a dose ranging from 2.0 to 3.0 micrograms/kg body weight one to five times a week.

57. The method of claim 41, wherein said interferon-gamma is administered parenterally or subcutaneously.

58. The method of claim 41, wherein said fibrotic interstitial lung disease is idiopathic pulmonary fibrosis (IPF), hypersensivity pneumonitis, scleroderma, Systemic Lupus Erythematosus, Rheumatoid Arthritis, Churg-Strauss syndrome, Wegener's granulomatosis, or Goodpasture Syndrome.

59. A kit comprising:
reagents for isolating polynucleotides from a biological sample;
a microarray comprising oligonucleotides and probes corresponding to genes which are over expressed or under expressed in interstitial lung disease;
a reference sample;
a microarray comprising oligonucleotides and probes useful for detecting infective agents;
interferon-gamma; and
glucocorticoids.

60. The kit of claim 59, wherein said overexpressed genes are selected from the group consisting of PTH-responsive osteosarcoma B1 protein, AF095771.1, matrix associated, actin dependent regulator of chromatin, subfamily f, member 1, AF231056.1, deleted in lung and esophageal cancer 1 (DLEC1), NM_{—}007337.1, major histocompatibility complex, class II, DQ beta 1, AW276186, SB classII histocompatibility antigen alpha-chain, AI128225, mucin 4, tracheobronchial, AJ242547.1, forkhead box J1 (FOXJ1), U69537.1, hypothetical protein FLJ21616, NM_{—}024567.1, neuronal specific transcription factor DAT1, AF258348.1, hematopoietic PBX-interacting protein, BF344265, proline oxidase homolog, AA074145, mucin 5, subtype B, tracheobronchial, AI697108, golgi membrane protein GP73, AF236056.1, ATP citrate lyase, U18197.1, NG22 protein, NM_{—}025257.1, cDNA DKFZp434A2322, AL137706.1, hepatocyte nuclear factor 3, alpha, U39840.1, major histocompatibility complex, class II, DQ alpha 1, X00452.1, myosin regulatory light chain 2, smooth muscle isoform, J02854.1, plexin B1, AV693216, pyruvate kinase, muscle, BC000481.1, tetraspanin TM4-C, AF133425.1, insulin-like growth factor binding protein 2 (36 kD), BC004312.1, FLJ13945 fis, clone Y79AA1000969, AU160041, hypothetical protein DKFZp586M1120, NM_{—}031294.1, CD24 signal transducer, L33930, hypothetical protein FLJ23571, NM_{—}025111.1, glutathione S-transferase M2 (muscle), M63509.1, cadherin 1, type 1, E-cadherin (epithelial), L08599.1, NTT5 protein, AF265578.1, lipocalin 2 (oncogene 24p3), NM_{—}005564.1, myotonic dystrophy kinase (DM kinase), L08835, uncoupling protein 2 (mitochondrial, proton carrier), U76367.1, dynein intermediate chain 2, NM_{—}023036.1, discoidin receptor tyrosine kinase isoform b, discoidin domain receptor family, member 1, NM_{—}001954.2, sperm associated antigen 6, AF079363.1, hypothetical protein FLJ23049, NM_{—}024687.1, nasopharyngeal epithelium specific protein 1, AF094758.1, nuclear receptor subfamily 4, group A, member 2, NM_{—}006186.1, hypothetical protein FLJ22215, BC003543.1, non-specific cross reacting antigen, M18728.1, amylase, alpha 1A; salivary (AMY1A), NM_{—}004038.1, carcinoembryonic antigen-related cell adhesion molecule 6 (non-specific cross reacting antigen), BC005008.1, glutathione S-transferase subunit 4 (EC 2.5.1.18), X08020.1, SH3-containing protein SH3GLB2, AF257319.1, KDEL (Lys-Asp-Glu-Leu) endoplasmic reticulum protein retention receptor 1, NM_{—}006801.1, anterior gradient 2 (Xenepus laevis) homolog, AF038451.1, sv7-MUC4 apomucin, mucin 4, tracheobronchial, AJ242547.1, stratifin, BC000329.1, connective tissue growth factor, M92934.1, cytochrome P450-IIB (hIIB3), M29873.1, filamin A, alpha (actin-binding protein-280), AW051856, membrane glycoprotein LIG-1, AB050468.1, E74-like factor 3 (ets domain transcription factor, epithelial-specific), U73844.1, elastin (supravalvular aortic stenosis, Williams-Beuren syndrome), M36860.1, ephrin receptor EPHA3, AF213459.1, fibrillin 1 (Marfan syndrome), L13923.1 discs, large (Drosophila) homolog 1, U13896.1, lysyl oxidase-like 1 (LOXL1), L21186.1, calumenin, U67280.1, male germ cell-associated kinase, NM_{—}005906.2, plectin 1, intermediate filament binding protein, 500 kD, Z54367, peroxisome biogenesis factor 1, AF026086.1, mast cell tryptase beta III, AF099143, ataxia-telangiectasia group D-associated protein, AF230388.1, hypothetical protein FLJ10921, NM_{—}018272.1, biglycan, BC002416.1, BLu protein, AC002481, CGI-92 protein, AF151850.1, adaptor-related protein complex 1, mu 2 subunit, BC003387.1, keratin 15, BC002641.1, B7 protein, U72508.1, S100 calcium-binding protein A2, BC002829.1, MUF1 protein, 80004953.1, cholesterol 25-hydroxylase, AF059214.1, cytidine monophosphate-N-acetylneuraminic acid hydroxy-lase (CMP-N-acetylneuraminate monooxygenase), AF074480.1, neuropilin 2, AF022859.1, Fas-interacting serinethreonine kinase 3, homeodomain-interacting protein kinase 3, AF305239.1, a disintegrin and metalloproteinase domain 28 (ADAM28), transcript variant 2, AF137334.1, cDNA DKFZp434A119, AW663632, complement component 6, J05064.1, complement component 6, J05064.1, cytokeratin 17, Z19574, wingless-type MMTV integration site family, member 5A, AI968085, matrix metalloproteinase 7 (matrilysin, uterine), BC003635.1, leiomodin 1 (smooth muscle), NM_{—}012134.1, Cip1-interacting zinc finger protein, AB030835.1, cyclin-dependent kinase inhibitor 1A (p21, Cip1), BC000275.1, integrin, alpha 7, AF032108.1, DKFZP586G011 protein, BG289527, fatty acid binding protein 6, ileal (gastrotropin), U19869.1, glutathione S-transferase M4, M96234.1, Ras-related associated with diabetes, L24564.1, claudin 3, AB000714.1, matrix metalloproteinase 10 (stromelysin 2), BC002591.1, fibulin 2, NM_{—}001998.1, serine threonine kinase 11 (STK11), AF035625, eukaryotic translation initiation factor 1A: AF000987.1, DEADH (Asp-Glu-Ala-AspHis) box polypeptide: AF000985.1, ribosomal protein S4: AF116711.1, ubiquitin specific protease 9: AF000986.2, SMC (mouse) homolog: U52191.1, myelin basic protein: L18865.1, S100 calcium-binding protein: NM_{—}005980.1, Jagged2 (JAG2): AF003521.1, latent transforming growth factor beta binding protein 4: NM_{—}003573.1, microtubule-associated protein, RPEB family, member 3: AB025186.1, Unknown (protein for MGC:2854): BC003629.1, clone=IMAGE-2406340: AI830563, AQP3 gene for aquaporine 3 (water channel): AB001325, cDNA DKFZp434A19, fenestrated-endothelial linked structure protein (FELS), PV1 protein (PLVAP): AF326591.1, LUNX protein; PLUNC (palate lung and nasal epithelium clone); tracheal epithelium enriched protein (LOC51297): AB024937.1, RAB, member of RAS oncogene family-like 2A: AF095350.1, chromosome 11 open reading frame 16: NM_{—}020643.1, hypothetical protein FLJ23049: NM_{—}024687.1, hypothetical protein FLJ11767: NM_{—}024593.1, dynein, axonemal, intermediate polypeptide: AF091619.1, brain specific protein (LOC51673): AF 132972.1, MUC4 apomucin, mucin 4, tracheobronchial: AJ242547.1, myotonin protein kinase (DM): M87313.1, cytokeratin 4: X07695.1, KIAA0362 gene, MCF.2 cell line derived transforming sequence-like: AB002360.1, cytokeratin 17: Z19574, LIM domain protein: BC003096.1, E74-like factor 3 (ets domain transcription factor, epithelial-specific): U73844.1, fatty acid binding protein 6, ileal (gastrotropin): U19869.1, sperm associated antigen 6: AF079363.1, eyes absent (Drosophila) homolog 2: U71207.1, phosphatidic acid phosphatase type 2C: BC002806.1, epoxide hydrolase 2, cytoplasmic: AF233334.1, tubulin, beta, 2: BC002783.1, heat shock 105 kD: D86956.1, heat shock 105 kD: D86956.1, villin 2 (ezrin): J05021.1, a disintegrin and metalloproteinase domain 28 (ADAM28), transcript variant 3: AF137335.1, deleted in lung and esophageal cancer 1: NM_{—}007337.1, arachidonate 15-lipoxygenase: NM_{—}001140.1, UDP glycosyltransferase 1 family, polypeptide A1: M57899.1, hypothetical protein PRO2834: AF 119903.1, lectin, galactoside-binding, soluble, 7 (galectin 7): L07769.1, B7 protein: U72508.1, ephrin receptor EPHA3: AF213459.1, forkhead box J1: U69537.1, BLu protein: U70824.1, aldehyde dehydrogenase 3 family, member A1: BC004370.1, NG22 protein: NM_{—}025257.1, small inducible cytokine subfamily A (Cys-Cys), member 14: NM_{—}004166.1, cysteine-rich protein 1 (intestinal): BC002738.1, putative GTP-binding protein similar to RAYRAB1C: BC000566.1, integrin, beta 4: NM_{—}000213.1, serine (or cysteine) proteinase inhibitor, clade B (ovalbumin), member 5 (SERPINB5): U04313.1, Ras-related associated with diabetes: L24564.1, hepatic leukemia factor: M95585.1, keratin 15: BC002641.1, nuclear receptor subfamily 4, group A, member 2: NM_{—}006186.1, sialyltransferase: U14550.1, glutathione S-transferase M2 (muscle): M63509.1, hypothetical protein FLJ13110: NM_{—}022912.1, S100 calcium-binding protein A2: NM_{—}005978.2, collagen, type VII, alpha 1 (epidermolysis bullosa, dystrophic, dominant and recessive): L02870.1, claudin 3: AB000714.1, insulin-like growth factor binding protein 6: BC003507.1, fibroblast growth factor receptor 2 (bacteria-expressed kinase, keratinocyte growth factor receptor, craniofacial dysostosis 1, Crouzon syndrome, Pfeiffer syndrome, Jackson-Weiss syndrome): M80634.1, insulin-like growth factor 1 receptor: NM_{—}000875.2, insulin-like growth factor binding protein 2 (36 kD): M35410.1, ataxia-telangiectasia group D-associated protein: AF230388.1, keratin 5 (epidermolysis bullosa simplex, Dowling-MearaKobnerWeber-Cockayne types): M21389.1, Duffy blood group: U01839.1, transforming growth factor β₁, NM_{—}000660

61. The kit of claim 59, wherein said underexpressed genes are selected from the group consisting of sorting nexin 10 (SNX10), AF121860.1, phospholipase A2, group IIA (platelets, synovial fluid), M22430.1, hemoglobin alpha-1 globin chain (HBA1), AF349571.1, macrophage scavenger receptor 1, AI299239, surfactant, pulmonary-associated protein C, BC005913.1, disintegrin protease (M12.219), NM_{—}014479.1, retinol-binding protein 4, interstitial, AF119868.1, major histocompatibility complex, class II, DR beta 3, M27635.1, adipose differentiation-related protein, BC005127.1, hemoglobin, delta, NM_{—}000519.2, beta-2-microglobulin, AW188940, hemoglobin, alpha 2, BC005931.1, protein C receptor, endothelial (EPCR), L35545.1, thymosin, beta 10, M92381.1, apolipoprotein C-I, W79394, hypothetical protein, AL133067.1, proteoglycan 4, (megakaryocyte stimulating factor, articular superficial zone protein), U70136.1, tetranectin (plasminogen-binding protein), NM_{—}003278.1, platelet factor 4, M25897.1, tissue factor pathway inhibitor 2, BC005330.1, fatty acid binding protein 4, adipocyte, BC003672.1, cathepsin B (CTSB), M14221.1, transmembrane 4 superfamily member 1, M90657.1, MHC class I HLA-B51 major histocompatibility complex, class I, E, M31183.1, endothelial PAS domain protein 1, U51626.1, Apo-2 ligand tumor necrosis factor (ligand) superfamily, member 10, U37518.1, haptoglobin-related protein, NM_{—}020995.1, Rho guanine exchange factor (GEF) 12, AF119898.1, major histocompatibility complex, class II, DR beta 5, M11867.1, interferon, gamma-inducible protein 30, AF097362.1, ferritin, light polypeptide, BG537190, prosaposin (variant Gaucher disease and variant metachromatic leukodystrophy), BC004275.1, calcium-binding protein A4 (calvasculin, metastasin,), NM_{—}002961.2, hemoglobin, beta, M25079.1, CDW52 antigen (CAMPATH-1 antigen), BC000644.1, aldehyde dehydrogenase 1 family, member A2, AB015226.1, cathepsin Z, AF032906.1, MHC HLA-B39 major histocompatibility complex, class I, B, L37880.1, major histocompatibility complex, class II, DQ alpha 1, M33906.1, fibroblast growth factor 9 (glia-activating factor), D14838.1, hemoglobin, alpha 2, AF097635.1, transferrin receptor (p90, CD71), BC001188.1, complement component 3 (C3), K02765.1, cDNA DKFZp564D066, AL050025.1, complement component 1, q subcomponent, beta polypeptide NM_{—}000491.2, small inducible cytokine subfamily A (Cys-Cys), member 18, pulmonary and activation-regulated, AB000221.1, reticulon 1, L10333.1, major histocompatibility complex, class II, DR beta 1, M33600.1, haptoglobin, L29394.1, acid phosphatase 5, tartrate resistant, J04430.1, cytochrome P450, subfamily XXVIIA (steroid 27-hydroxylase, cerebrotendinous xanthomatosis), polypeptide 1, M62401.1, CD36 antigen (collagen type I receptor, thrombospondin receptor), M24795.1, calbindin 2, (29 kD, calretinin), NM_{—}001740.2, alpha-2-HS-glycoprotein, BG538564, cDNA DKFZp564A132, AL049963.1, fibronectin 1, AF130095.1, phosphodiesterase 4C, cAMP-specific, NM_{—}000923.1, transcription factor 7 (T-cell specific, HMG-box), NM_{—}003202.1, found in inflammatory zone 3 (FIZZ3), AF323081.1, claudin 15, NM_{—}014343.1, carboxypeptidase B1 (tissue), M81057.1, hypothetical protein FLJ14054, NM_{—}024563.1, bone marrow stromal cell antigen 1, D21878.1, interleukin 7 receptor, M29696.1, procollagen C-endopeptidase enhancer 2, AF098269.1, calcium-binding protein A8 (calgranulin A), AW238654, cDNA DKFZp564D193, AL049252.1, major histocompatibility complex, class II, DP beta 1, J03041.1, human leukocyte antigen C alpha chain, major histocompatibility complex, class I, C, AK024836.1, BCM-like membrane protein precursor, AF144235.1, CD14 antigen, M86511.1, pulmonary surfactant protein (SP5), J03553, signal transducer and activator of transcription 1, 91 kD, BC002704.1, Wilms tumor 1 (WT1), transcript variant D, NM_{—}024424.1, annexin A8, BC004376.1, macrophage receptor with collagenous structure, MARCO, AF035819.1, surfactant, pulmonary-associated protein A2, NM_{—}006926.1, solute carrier family 6 (neurotransmitter transporter, serotonin), member 4, L05568.1, chitinase 1 (chitotriosidase), U29615.1, lung type-I cell membrane-associated glycoprotein, AU154455, fibronectin leucine rich transmembrane protein 2, AB007865.1, gamma-aminobutyric acid (GABA) A receptor, alpha 5, BF966183, hypothetical protein FLJ12983, NM_{—}024856.1, sialophorin (gpL115, leukosialin, CD43), J04536.1, cerebellar degeneration-related protein (34 kD), M16965.1, hydroxyacid oxidase 2 (long chain), hydroxy-delta-5-steroid dehydrogenase (3 beta- and steroid delta-isomerase 2), AL359553, CLONE=IMAGE:1032795=Hs.83623 nuclear receptor subfamily 1, group I, member 3, small inducible cytokine subfamily C, member 2: NM_{—}003175.1, hypothetical protein similar to swine acylneuraminate lyase: NM_{—}030769.1, fibrinogen, gamma polypeptide: AF118092.1, thrombospondin 1: NM_{—}003246.1, SAM domain, SH3 domain and nuclear localisation signals, 1: AF222927.1, chitinase 3-like 1 (cartilage glycoprotein-39): M80927.1, cathepsin Z: AF032906.1, CLONE=IMAGE:3579023 collagen, type XIV, alpha 1 (undulin), chloride intracellular channel 2: NM_{—}001289.2, monokine induced by gamma interferon: NM_{—}002416.1, KIAA0433 protein: NM_{—}015216.1, tumor necrosis factor, alpha-induced protein 6: NM_{—}007115.1, signal transducer and activator of transcription 1, 91 kD: BC002704.1, thrombospondin 2: L12350.1, collagen, type IV, alpha 3 (Goodpasture antigen): NM_{—}000091.1, integrin, beta-like 1 (with EGF-like repeat domains): AF072752.1, diubiquitin: NM_{—}006398.1, protease, cysteine, 1 (legnmain): D55696.1, cytochrome P450, subfamily I (dioxin-inducible), polypeptide 1 (glaucoma 3, primary infantile): U03688.1, integrin, alpha 1: X68742.1, GABA-B receptor, G protein-coupled receptor 51: AF056085.1, KIAA1199 protein: AB033025.1, collagen, type V, alpha 2: NM_{—}000393.1, interleukin 13 receptor, alpha 2: U70981.1, translocase of inner mitochondrial membrane 8 (yeast) homolog A: BC005236.1, steroid sulfatase (microsomal), arylsulfatase C, isozyme S: M16505.1, CLONE=IMAGE:1982571 ATPase, H+ transporting, lysosomal (vacuolar proton pump) 9 kD, proteoglycan 4, (megakaryocyte stimulating factor, articular superficial zone protein): U70136.1, nidogen (enactin): M30269.1, KIAA1598 protein: AU157109, vascular cell adhesion molecule 1: M60335.1, guanylate binding protein 1, interferon-inducible, 67 kD: BC002666.1, cDNA DKFZp586E1124, apolipoprotein H (beta-2-glycoprotein I): M62839.1, ribosomal protein L37a: BE857772, cDNA DKFZp564A132, cathepsin S: M86553.1, a disintegrin and metalloproteinase domain 9 (meltrin gamma) (ADAM9): U41766.1, zinc finger protein 331: AF272148.1, lysosomal-associated membrane protein 2: J04183.1, carboxypeptidase M: NM_{—}001874.1, collagen, type I, alpha 2: NM_{—}000089.1, P311 protein: U36189.1, KIAA0372 gene product: AB002370.1, Human T cell-specific protein RANTES: M21121, interferon-gamma-inducible indoleamine 2,3-dioxygenase (IDO): M34455.1, hypothetical protein FLJ10430: NM_{—}018092.1, transcription factor ISGF-3: M97935, interleukin 1 receptor-like 1 (IL1RL1): NM_{—}003856.1, putative alpha chemokine (H174), small inducible cytokine subfamily B (Cys-X-Cys), member II: AF002985.1, small inducible cytokine subfamily B (Cys-X-Cys), member 10: NM_{—}001565.1, mesoderm specific transcript (mouse) homolog: BC002413.1, carboxypeptidase B-like protein: AB011969.1, CD2 antigen (p50), sheep red blood cell receptor: M16445, interferon, alpha-inducible protein (clone IFI-6-16): NM_{—}022872.1, RAS guanyl releasing protein 1 (calcium and DAG-regulated): AF081195.1, lipase, endothelial: AF118767.1, fatty-acid-Coenzyme A ligase, long-chain 4: NM 022977.1, klotho: AB005142.1, chondroitin sulfate proteoglycan 2 (versican): NM_{—}004385.1, hypothetical protein, expressed in osteoblast: AB000115.1, CD14 antigen: M86511.1, CGI-83 protein: BC000878.1, leucine aminopeptidase: AF061738.1, UDP-Gal:betaGlcNAc beta 1,3-galactosyltransferase, polypeptide 3: AB050855.1, protocadherin alpha 12: AF152308.1, neuroglycan C: AF059274, neuroligin: AI338338, HSPC156 protein: AF161505.1, hypothetical protein FLJ13310: NM_{—}025118.1, eosinophil chemotactic cytokine (TSA1902): AB025008.1, aminopeptidase: AF191545.1, semaphorin sem2: AB029496.1, protocadherin 12: AF231025.1, X transporter protein 3: NM_{—}020208.1, transmembrane 4 superfamily member (tetraspan NET-2): AF124522.1, hypothetical protein FLJ10970: NM_{—}018286.1, perforin 1 (pore forming protein): M28393.1, natural killer cell group 7 sequence: NM_{—}005601.1, hypothetical protein DKFZp761N09121: BF435376, integrin, alpha 4 (antigen CD49D, alpha 4 subunit of VLA-4 receptor): BG532690, chitinase 3-like 2: U58515.1, FYN oncogene: N20923, relaxin 1 (H1): BC005956.1, hydroxyprostaglandin dehydrogenase 15-(NAD): U63296.1, sulfotransferase family, cytosolic, 1C, member 1: AF186254.1, TGF-b superfamily receptor type I: L17075.1, granzyme B (granzyme 2, cytotoxic T-lymphocyte-associated serine esterase 1): M36118.1, cystatin F (leukocystatin): AF031824.1, regulator of G protein signaling-Z (RGSZ1): AF060877.2, clone MGC:12387: M16942.1, zinc-alpha2-glycoprotein: D90427.1, BCG induced integral membrane protein BIGMo-103: AB040120.1, nectin-like protein 2 (NECL2): AF132811.1, CD209 antigen-like: AB015629.1, solute carrier family 6 (neurotransmitter transporter, serotonin), member 4: L05568.1, MAD (mothers against decapentaplegic, Drosophila) homolog 6: U59914.1, latrophilin: AF104939.1, platelet factor 4: M25897.1, calcitonin receptor-like: L76380.1, matrilin 3: NM_{—}002381.2, solute carrier family 14 (urea transporter), member 1 (Kidd blood group): U35735.1, interleukin 7 receptor: M29696.1, MAP kinase kinase 6 (MKK6), mitogen-activated protein kinase kinase 6: U39656.1, protocadherin 17: AF029343.1, granulysin: NM_{—}006433.2, interferon-stimulated protein, 15 kDa (ISG15): M13755.1, cadherin 5, type 2, VE-cadherin (vascularepithelium): U84722.1, thrombomodulin: M16552.1, interferon, alpha-inducible protein 27: NM_{—}005532.1, interferon-gamma, X87308.

62. The kit of claim 59, wherein said reference sample comprises a database of gene expression levels, in a healthy individual, for the genes overexpressed or underexpressed in interstitial lung disease.

63. The kit of claim 59, wherein said infective agents are bacteria, viruses, yeast and fungi.

64. The kit of claim 59, wherein said interferon-gamma is pegylated.
